(19) United States
(12) Patent Application Publication
(10) Pub. No.: US 2025/0032624 A1
(43) Pub. Date: Jan. 30, 2025

(54) SMALL MOLECULE DEGRADATION METHODS FOR TREATING ALS/FTD

(71) Applicant: University of Florida Research Foundation, Incorporated, Gainesville, FL (US)

(72) Inventor: Matthew D. Disney, Gainesville, FL (US)

(21) Appl. No.: 18/704,712

(22) PCT Filed: Oct. 27, 2022
(86) PCT No.: PCT/US2022/078830

§ 371 Date: Apr. 25, 2024

Related U.S. Application Data
(60) Provisional application No. 63/272,526, filed on Oct. 27, 2021.

(51) Int. Cl. A61K 47/55; A61K 47/60; A61P 25/28

(52) U.S. Cl. CPC A61K 47/55; A61K 47/60; A61P 25/28

(57) ABSTRACT

Described are small molecule embodiments, ALS compounds, that bind with the r(G₄C₂)^{exp} RNA repeat expansion present in chromosome 9 open reading frame 72 involved in amyotrophic lateral sclerosis and frontotemporal dementia (ALS/FTD). These ALS compounds comprise a pyridocarbazole moiety having at least one substituent and an RNase-recruiting moiety linked to the pyridocarbazole moiety by a polyethylene glycol group.

### STATEMENT OF GOVERNMENT SUPPORT

This invention was made with government support under grant numbers NS096898, NS099114 and NS116846 awarded by the National Institutes of Health. The government has certain rights in the invention.

### REFERENCE TO AN ELECTRONIC SEQUENCE LISTING

The contents of the electronic sequence listing (U120270103WO00-SEQ-JDH.xml; Size: 55,627 bytes; and Date of Creation: Oct. 27, 2022) is herein incorporated by reference in its entirety.

### BACKGROUND

RNA repeat expansion disorders are defined by short repeating sequences of RNA and are responsible for over 30 human diseases, most of which are neurodegenerative in nature.¹ Among these disorders, several are considered uncurable by today's standards. The patho-mechanisms of these RNAs stem from the formation of disease-specific RNA structures, most commonly hairpin structures, which form from the repeating RNA and are absent in transcripts lacking these repeats.² These structures interfere with canonical cellular biology, affecting processes such as pre-mRNA processing, RNA/protein complex formation, and translation.^{2,3}

One such disorder is C9orf72-associated amyotrophic lateral sclerosis (ALS) and frontotemporal dementia (FTD), collectively referred to as c9ALS/FTD. The genetic involvement in this disorder can be traced to the hexanucleotide repeat expansion, GGGGCC [r(G₄C2)^{exp}], found in the first intron of chromosome 9 open reading frame 72 (C9orf72).^{4,5} This r(G₄C₂) repeat expansion is responsible for the majority of cases of familial c9ALS/FTD making this repeat the leading genetic cause of c9ALS/FTD.^{4,5} Thus, novel therapeutic modalities to target and reduce this repeat expansion are in high demand. While antisense oligonucleotides (ASOs) currently offer the most advanced therapeutic intervention against r(G₄C₂)^{exp}, ASOs generally have low tissue penetrance and distribution.⁶⁻⁹ Additionally, ASOs have to be administered intrathecally, increasing the risk and discomfort to the patient upon treatment.¹⁰ Thus, small molecules offer an attractive alternative to ASOs as their low molecular weight favors them to be bio-orally available.^{11,12}

The r(G₄C₂)^{exp}, consisting of repeat lengths typically >30, contributes to c9ALS/FTD pathology via two main mechanisms; 1) sequestration of RNA-binding proteins, specifically heterogenous nuclear ribonucleoprotein H (hnRNP H); and 2) initiation of repeat-associated non-AUG (RAN) translation.^{4,13-19} The hairpin structure formed by the hexanucleotide repeats, creates 1×1 GG internal loops that sequester hnRNP H, leading to the formation of nuclear foci and disruption of pre-mRNA processing (i.e. retention of intron 1).^{11,19,20} These foci in turn disrupt nucleocytoplasmic transport further contributing to the pathology of c9ALS/FTD. In addition to the toxicity caused by these mechanisms, RAN translation produces toxic dipeptide repeat (DPR) proteins, contributing to neuronal cell death.^{19,21,22}

Therefore, an objective of the present invention is development of a small molecule treatment that would reduce the abundance of the r(G₄C₂)^{exp} in cells. Achievement of this objective provides an attractive therapeutic modality for alleviating downstream pathologies of c9ALS/FTD.

### SUMMARY

The present invention is directed to methods for treatment of ALS/FTD. Aspects of the method relate to embodiments of an ALS compound which is capable of binding with and inducing enzymatic degradation of the RNA sequence transcribed from the microsatellite G₄C₂ repeat in the C9ORF72 genotype. Embodiments of the ALS compound comprise in part a polycyclic heteroaromatic compound, specifically a pyridocarbazole moiety. These embodiments further comprise at least a substituent bound to the pyridocarbazole moiety comprising a linker group carrying an RNase recruiting moiety. In particular, embodiments of the ALS compound comprise a pyridocarbazole RNase recruiter compound of Formula I in which R is hydrogen or a C1-C3 alkyl, preferably methyl or hydrogen and more preferably hydrogen, n is and integer of 1 to 6, preferably 4 and Y is —COO— or —CH₂O—, preferably —CH₂O—.

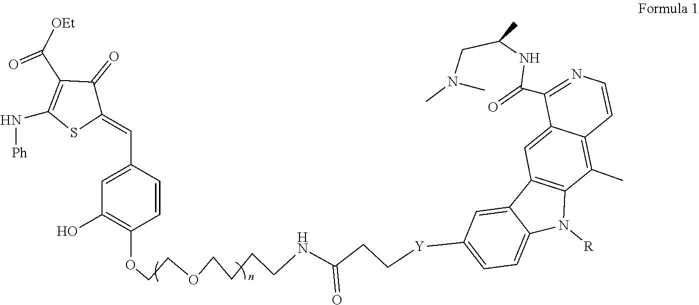

The ALS compound of Formula I may be considered as having two functions: the r(G₄C₂) repeat binding function and the RNase recruiting function. The binding function is attributed to the carbazole core of Formula II in which X may be hydroxyl.

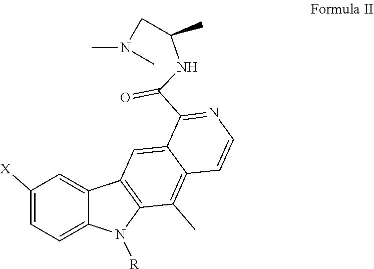

The RNase recruiting function is attributed to the phenoxy thiophenone moiety of Formula IIIA. The active form of Formula IIIA is provided when R″ is hydrogen and R′ is an alkyl or other non-hydrogen group. The inactive form of Formula IIIA is provided when R″ is alkyl or other non-hydrogen group and R′ is hydrogen. The phenoxythiophenone moiety of Formula IIIA is linked at its R′ position to the carbazole core of Formula II at X through a PEG-amide linker of Formula IV. The combination of Formula IIIA and Formula IV yields the RNase-linker of Formula IIIB.

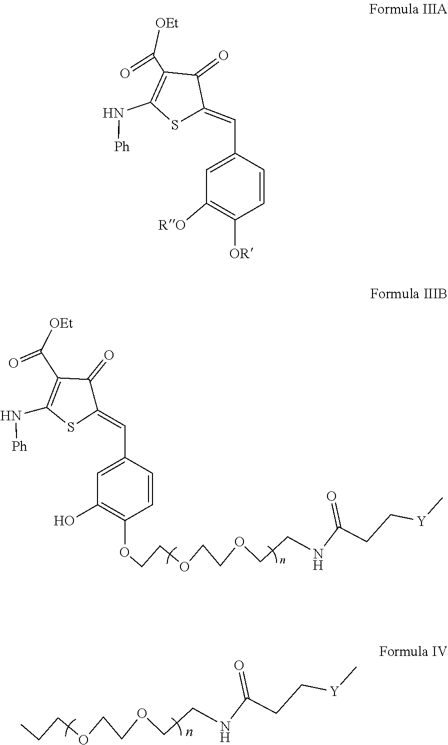

While all Y, R and n versions of the ALS compound of Formula I provide the biological activities herein described, preferred compositional embodiments of the invention include the ALS compound of Formula I in which Y is —CH₂O—, R is hydrogen and n is 2. The pharmaceutically acceptable salts of the compositional embodiments of Formula I are also included as aspects of the invention.

Additional embodiments include methods for complexing and/or binding the ALS compound with the RNA repeat r(G₄C2)^{exp} which is r(G₄C2)ₘ with m as an integer designator of 24-1,000's. These embodiments include methods for complexing and/or binding an abnormal number of RNA repeats in which m is at least 100, preferably at least 200, and more preferably at least 500 to at least 1000. For these embodiments the RNA repeat sequence is at least an RNA hairpin structure.

Further embodiments include methods in which the RNA repeat is present in cells such as but not limited to cell cultures, HEK293T cells transfected to express the RNA repeat expansion, ALS patient-derived cells, lymphoblastoid cells, induced pluripotent stem cells (c9 iPSCs cells) and iPSC-derived spinal neuron cells (c9 iPSNs). The RNA repeat may also be present as c9ALS/FTD BAC cells of a transgenic mouse. The RNA repeat r(G₄C2)^{exp} may be present or may be transcribed in these cells when the cells contain chromosome 9 open reading frame 72 known as C9orf72 and r(G₄C2)^{exp} as an abnormal repeat present in intron 1 of C9orf72.

Embodiments of the methods also enable the ALS compound as Formula I to decrease and/or inhibit RAN translation of r(G₄C2)^{exp} RNA in cells including but not limited to those mentioned above. Further, these methods preferably do not inhibit transcription of the C9orf 72.

Embodiments according to the invention further include pharmaceutical compositions comprising an ALS compound and a pharmaceutically acceptable carrier. Preferably, the ALS compound comprises Formula I. More preferably, the ALS compound of Formula I has designation n as 4. Preferably, the pharmaceutical composition comprises an effective amount, preferably an effective dose of the ALS compound for treatment of ALS/FTD disease.

Embodiments according to the invention also include a method of treatment of patients suffering from ALS/FTD. These embodiments comprise administration of an effective amount of an ALS compound of Formula I, preferably with designation n as 4. These embodiments also comprise administration of a pharmaceutically acceptable composition with an effective amount or effective dose of the ALS compound of Formula I. Preferably routes of administration include oral, intraperitoneal (ip), intravenous (iv), intramuscular (im), subcutaneous (SC), oral, rectal, vaginal, intrathecal, and/or intradermal. Preferably, the disease is amyotrophic lateral sclerosis.

Additional embodiments according to the invention include methods for treatment of other diseases caused by r(G₄C₂) RNA repeat expansions. While ALS and FTD are two extremes of the disease spectrum associated with this repeat expansion, the disease spectrum includes a range of neuropsychological deficits such as cognitive impairment, behavioral impairment, and several other manifestations. All of these diseases may be treated as described herein for treatment of ALS/FTD and the ALS-FTD disease spectrum. See for example, M. B. Leko, et. al., Behav, Neurol., v. 2019, Jan. 15, 2019, 2909168.

### BRIEF DESCRIPTION OF DRAWINGS

FIGS. 1A-1C depicts how the monomeric small molecule targets the r(G₄C₂)^{exp}. FIG. 1A shows the structure of the small molecule binder compound 1 (labeled as 1, compound of Formula II), which binds the r(G₄C₂)^{exp}. Formula II (Compound 1) was previously reported to selectively binding to the G:G loop. FIG. 1B shows the structures of the ALS compound of Formula I (hereinafter ALSFI, labeled as 2, also called RIBOTAC 2 herein, hereinafter compound 2) and a negative control (compound 3 in which the RNase recruiting moiety of Formula IIIA is bound to linker moiety Formula IV at R″ instead of R′). Use of the R″ position of Formula IIIA as an attachment for the linker Formula IV provides a less active regioisomer of the RNase L-recruiting module Formula IIIA, the recruiter moiety. FIG. 1C is a schematic of the mechanism of action of the ALSFI and the lack of action when compound 3 (use of inactive binding site R″ of Formula IIIA).

FIGS. 2A-2E depict how the ALSFI (labeled as 2) diminishes c9ALS/FTD pathologies in patient-derived lymphoblastoid cells. FIG. 2A is a schematic representation of repeat-associated non-AUG (RAN) translation producing toxic dipeptide repeat proteins (DPRs), which occurs when the r(G₄C₂)^{exp} is present in intron 1 of C9orf72. FIG. 2B shows ALSFI (labeled as 2) decreases poly(GP) abundance dose-dependently, as measured by an electroluminescent sandwich immunoassay, in c9ALS patient-derived lymphoblastoid cells (LCLs) (n=3 C9orf72 LCLs, 3 replicated per line). FIG. 2C depicts a schematic representation of the C9orf72 alternative splicing isoforms that result from retention of C9orf72 intron 1. Arrows indicate the location of the three RT-qPCR primer sets used throughout these studies (intron 1 primers, exon 2-3 primers, and exon 1b primers). FIG. 2D shows ALSFI (labeled as 2) decreases C9orf72 intron 1 abundance dose-dependently, as measured by RT-qPCR (n=3 C9orf72 LCLs lines, 3 replicated per line). FIG. 2E shows the competitive binding between the ALSFI (labeled as 2) and the carbazole core binder of Formula II (labeled as 1, i.e., compound 1 of FIG. 1A) validate that ALSFI and the carbazole core binder of Formula II share the same target in cells, as measured by RT-qPCR (n=1 C9orf72 LCLs, 3 replicated per line). Vehicle indicates 0.1% (v/v) DMSO. *P<0.05, **P<0.001, ***P<0.0001, ****P<0.0001 as determined by a One-Way ANOVA with multiple comparisons (FIGS. 2B, 2D & 2E). Error bars represent SD.

FIGS. 3A-3F illustrate how ALSFI (labeled as 2) diminishes c9ALS/FTD pathologies in patient-derived iPSCs and differentiated motor neurons. For all panels, vehicle indicates 0.1% (v/v) DMSO. *P<0.05, **P<0.001, ***P<0.0001, ****P<0.0001 as determined by a One-Way ANOVA with multiple comparisons. Error bars indicate SD. FIG. 3A shows ALSFI (labeled as 2) decreases poly(GP) abundance dose-dependently, as measured by an electrochemical luminescent sandwich immunoassay, in c9ALS patient-derived iPSCs (n=4 C9orf72 iPSC lines, 3 replicates per line). FIG. 3B shows ALSFI (labeled as 2) decreases C9orf72 intron 1 abundance dose-dependently, as measured by RT-qPCR (n=4 C9orf72 iPSC lines, 3 replicates per line). FIG. 3C shows ALSFI (labeled as 2) decreases C9orf72 exon 2-3 abundance dose-dependently, as measured by RT-qPCR (n=4 C9orf72 iPSC lines, 3 replicates per line). FIG. 3D shows ALSFI (labeled as 2) decreases poly(GP) abundance dose-dependently, as measured by AN ELECTROCHEMICAL LUMINESCENT SANDWICH IMMUNOASSAY, in c9ALS motor neurons differentiated from patient-derived iPSCs (n=1 C9orf72 motor neuron line, 3 replicates per line). FIG. 3E ALSFI (labeled as 2) decreases C9orf72 intron 1 abundance dose-dependently, as measured by RT-qPCR (n=1 C9orf72 motor neuron line, 3 replicates per line). FIG. 3F ALSFI (labeled as 2) decreases C9orf72 exon 2-3 abundance dose-dependently, as measured by RT-qPCR (n=1 C9orf72 motor neuron line, 3 replicates per line).

FIGS. 4A-4E disclose that ALSFI (labeled as 2) clears the r(G₄C₂)^{exp} from c9ALS patient-derived iPSCs via a unique mechanism of degradation. Vehicle indicates 0.1% (v/v) DMSO. *P<0.05, **P<0.001, ***P<0.0001, ****P<0.0001 as determined by unpaired t-tests with Welch's correction (FIGS. 4B-4E). Error bars represent SD. FIG. 4A is a schematic (at left side) of the nuclear exosome which is responsible for endogenous 3′ to 5′ RNA degradation; and a schematic (at right side) of RNase L-induced cleavage of the r(G₄C₂)^{exp}. FIG. 4B is a graph showing how the knock down of the activity of nuclear exosome (hRRP6), RNase L or hRRP6 and RNase L together by targeted siRNA treatment results in an ablation of C9orf72 intron 1 decay when co-treated with ALSFI (labeled as 2) (n=1 iPSC line, 5 replicates per line). FIG. 4C shows how the knock down of the activity of nuclear exosome (hRRP6) by targeted siRNA treatment, has no effect on C9orf72 exon 2-3 abundance when co-treated with ALSFI (labeled as 2), as measured by RT-qPCR using primers for intron 1. Knock down of the activity of RNase L or hRRP6 and RNase L together by targeted siRNA treatment, results in ablation C9orf72 cleavage, as measured by RT-qPCR using primers for exon 2-3 (n=1 iPSC line, 5 replicates per line). FIG. 4D shows how Knocking down the activity of XRN1 by targeted siRNA treatment results in an ablation of C9orf72 intron 1 decay when co-treated with ALSFI (labeled as 2). Knocking down XRN2 activity with targeted siRNA treatment has no effect on C9orf72 intron 1 decay when co-treated with RIBOTAC 2 (n=1 iPSC line, 5 replicates per line). FIG. 4E shows how knock down of XRN1 activity by targeted siRNA treatment results in an ablation of C9orf72 degradation when co-treated with ALSFI. Knockdown of XRN2 with targeted siRNA treatment has no effect on C9orf72 degradation when co-treated with ALSFI, as measured by RT-qPCR using primers for exon 2-3 (n=1 iPSC line, 5 replicates per line).

FIGS. 5A-5H show how ALSFI diminishes c9ALS/FTD pathologies in a BAC transgenic mouse model (+/+PWR500) of the r(G₄C₂)^{exp}. For all panels, vehicle indicates an injection of 1% DMSO 99% H₂O. *P<0.05, **P<0.001, ***P<0.0001, as determined by an unpaired t-test with Welch's correction. Error bars indicate SD. FIG. 5A shows treatment with ALSFI (labeled as 2) decreases C9orf72 intron 1 abundance in the +/+PWR500 mouse model of c9ALS, as measured by RT-qPCR using primers specific for intron 1 (n=5+/+PWR500 mice). FIG. 5B shows treatment with ALSFI (labeled as 2) decreases C9orf72 exon 2-3 abundance in the +/+PWR500 mouse model of c9ALS, as measured by RT-qPCR using primers specific for the exon 2-3 junction (n=5+/+PWR500 mice). FIG. 5C shows treatment with ALSFI (labeled as 2) has no effect on C9orf72 exon 1b abundance, in the +/+PWR500 mouse model of c9ALS, as measured by RT-qPCR using primers specific for the exon 1b (n=5+/+PWR500 mice). FIG. 5D shows treatment with ALSFI (labeled as 2) has no effect on C9orf72 exon 1b—exon 2 junction, in the +/+PWR500 mouse model of c9ALS, as measured by RT-qPCR (n=5+/+PWR500 mice). FIG. 5E shows treatment with ALSFI (labeled as 2) decreases poly(GP) abundance in the +/+PWR500 mouse model of c9ALS, as measured by an electroluminescent sandwich immunoassay (n=5+/+PWR500 mice). FIG. 5F shows the treatment with ALS compound Formula I (labeled as 2) has no effect on 3-actin abundance in the +/+PWR500 mouse model of c9ALS, as measured by an electroluminescent sandwich immunoassay (n=5+/+PWR500 mice). FIG. 5G shows Poly(GP) is almost indetectable in a wild type (WT; −/−PWR500) control mouse line, as measured by an electroluminescent sandwich immunoassay (n=3 WT mice). FIG. 5H shows treatment with ALSFI (labeled as 2) has no effect on β-actin abundance in the WT control mouse model, as measured by electroluminescent sandwich immunoassay (n=3−/−WT mice).

FIGS. 6A-6F show how ALSFI (labeled as 2) reduces toxic protein and RNA aggregates in +/+PWR500 mice. Vehicle indicates an injection of 1% DMSO 99% H₂O. *P<0.05, **P<0.001, ***P<0.0001, as determined by an unpaired t-test with Welch's correction. Error bars indicate SD. FIG. 6A shows immunohistochemistry (IHC) staining of the cortex of +/+PWR500 mice treated with vehicle and ALSFI (labeled as 2). Arrows point to protein aggregates. Representative images shown (n=3 cortex slices per mouse, 5 images quantified per slide). FIG. 6B shows treatment with ALSFI (labeled as 2) significantly reduces the number of poly(GP) aggregates per cell (n=3+/+PWR500 mice treated with vehicle and n=3+/+PWR500 mice treated with 2). FIG. 6C shows treatment with ALSFI (labeled as 2) significantly reduces the number of poly(GA) aggregates per cell (n=3+/+PWR500 mice treated with vehicle and n=3+/+PWR500 mice treated with 2). FIG. 6C shows treatment with ALSFI (labeled as 2) significantly reduces the number of TDP-43 aggregates per cell (n=3+/+PWR500 mice treated with vehicle and n=3+/+PWR500 mice treated with 2). FIG. 6D shows fluorescent in situ hybridization images indicating r(G₄C₂)^{exp}-containing RNA foci in the cortex of +/+PWR500 mice. FIG. 6F shows treatment with ALSFI (labeled as 2) significantly decreases the number of RNA foci per nuclei (n=4 +/+PWR500 mice treated with vehicle and n=4+/+PWR500 mice treated with 2).

FIG. 7 is a bar graph of the relative melanoma differentiation-associated protein 5 (MDA5) mRNA levels, a marker of immune-related inflammation, at various doses of ALSFI, as compared to 2′-5′poly(A), which induces a viral immune response.

FIGS. 8A-8B show ALSFI inhibits RAN translation in a transfected cellular model. FIG. 8A is a schematic of co-transfection of a No ATG-d(G₄C₂)₆₆-GFP (SEQ ID NO: 45) plasmid and an ATG-mCherry plasmid into HEK293T cells allows for RAN translation to be measured based on the GFP signal. The mCherry signal measures canonical translation. FIG. 8B shows ALSFI (labeled as 2) inhibits RAN translation dose-dependently in the transfected system, similar to an ASO targeting the r(G₄C₂)^{exp} (n=3 biological replicates). **P<0.001, ***P<0.0001, ****P<0.0001 as determined by a One-Way ANOVA. Error bars represent SD.

FIGS. 9A-9D show that ALSFI (labeled as 2) selectively binds r(G₄C₂)₈ (SEQ ID NO: 1) in vitro. Error bars indicate SD for all panels. FIG. 9A (Left) shows binding of ALSFI to target sequence r(G₄C₂)₈ (SEQ ID NO: 1), and control sequences d(G₄C₂)₈ (SEQ ID NO: 1) and r(GGCC)₈ (SEQ ID NO: 47), reported by K_{d} measured by microscale thermophoresis. Right: Schematic representation of oligos used for MST binding assays. FIG. 9B is the representative binding curve of ALSFI to r(G₄C₂)₈ (SEQ ID NO: 1) (n=2 replicate experiments each run in technical triplicates). FIG. 9C is the representative binding curve of ALSFI to d(G₄C₂)₈ (SEQ ID NO: 1) (n=2 replicate experiments each run in technical triplicates). FIG. 9D shows the representative binding curve of ALSFI to r(GGCC)₈ (SEQ ID NO: 47) (n=2 replicate experiments each run in technical triplicates).

FIGS. 10A-10c show that ALSFI (labeled in FIG. 2) cleaves r(G₄C₂)₈ (SEQ ID NO: 1) in vitro. *P<0.05 as determined by a One-Way ANOVA. Error bars indicate SD. FIG. 10 shows treatment with ALSFI (labeled as 2) cleaves 5′-end labeled (³²P) r(G₄C₂)₈ (SEQ ID NO: 1) in vitro. Colored boxes indicate sites of cleavage by ALSFI. FIG. 10B shows I quantification of cleavage gel in FIG. 10A (n=2 replicate experiments). FIG. 10C shows ALSFI-mediated cleavage sites mapped to the r(G₄C₂)₈ (SEQ ID NO: 1) hairpin structure.

FIGS. 11A-11C present bar graphs of cellular viability of ALSFI (labeled as 2). Vehicle indicates 0.1% (v/v) DMSO. Error bars indicate SD. FIG. 11A is a bar graph showing that ALSFI shows no toxicity in LCLs up to 1000 nM (n=1 LCL line, 3 replicates per line). FIG. 11B is a bar graph showing that ALSFI shows no toxicity in c9ALS patient-derived iPSCs up to 1000 nM (n=1 C9orf72 IPSC line, 4 replicates per line). FIG. 11C is a bar graph showing that ALSFI shows no toxicity in differentiated IPSNs up to 1000 nM (n=1 C9orf72 motor neuron line, 4 replicates per line).

FIGS. 12A-12G are bar graphs showing bioeffect of ALSFI (labeled as 2 on graphs) on healthy patients. Vehicle indicates 0.1% (v/v) DMSO. Error bars indicate SD. FIG. 12A shows ALSFI has no effect on C9orf72 intron 1 abundance in LCLs derived from healthy donors, as measured by rt-QPCR (n=1 healthy LCL line, 3 replicates per line). FIG. 12B shows ALSFI has no effect on C9orf72 exon 1b abundance in c9ALS patient-derived iPSCs, as measured by rt-QPCR (n=4 C9orf72 IPSC lines, 3 replicates each). FIG. 12C shows ALSFI has no effect on C9orf72 intron 1 abundance in iPSCs derived from healthy donors, as measured by rt-QPCR (n=3 healtHY IPSC lines, 3 replicates per line). FIG. 12D shows ALSFI has no effect on C9orf72 exon 2-3 abundance in iPSCs derived from healthy donors, as measured by rt-QPCR (n=3 healtHY IPSC lines, 3 replicates per line). FIG. 12E shows ALSFI has no effect on C9orf72 exon 1b abundance in iPSCs derived from healthy donors, as measured by rt-QPCR (n=3 healtHY IPSC lines, 3 replicates per line). FIG. 12F shows the control RNase Formula IIIA has no effect on C9orf72 intron 1 abundance, as measured by rt-QPCR (n=2 C9orf72 IPSC lines, 3 replicates each). FIG. 12G shows ALSFI only affects the abundance of one short-r(G₄C₂)exp-containing transcript (SOCS1) compared to a (G₄C₂)^{exp}-targeting ASO which affects the abundance of four short-r(G₄C₂)^{exp}-containing transcripts (SOCS1, XYLT1, Rab-40C, and RNA BP 10), as measured by rt-QPCR using primers specific for each repeat-containing transcript (n=1 C9orf72 IPSC line, 3 replicates per line).

FIG. 13 is a bar graph showing the relative C9orf72 intron 1 MRNA levels over time after treatment with ALSFI.

FIGS. 14A-14F show graphs of treatment OF IPSC's with ALSFI (labeled as Ribotac 2). Vehicle indicates 0.1% (v/v) DMSO. *P<0.05, **P<0.001, ***P<0.0001, ****P<0.0001 as determined by a One-Way ANOVA with multiple comparisons compared to the vehicle-treated sample for each transcript primer set. Error bars indicate SD. FIG. 14A is a bar graph showing C9orf72 intron 1 to exon 2 ratio as measured by RNA-seq in c9ALS patient-derived iPSCs (n=1 C9orf72 IPSC line, 3 replicates per line). FIG. 14B is a bar graph showing RNA-seq relative read counts per treatment group in c9ALS patient-derived iPSCs. FIG. 14C is a plot showing the log₂(Fold Change) vs Gene Abundance as measured by RNA-seq in c9ALS patient-derived iPSCs. The red dots indicate genes significantly up or down regulated transcriptome wide. The blue dot represented C9orf72. FIG. 14D is a bar graph showing RNA-seq relative read counts per treatment group in healthy donor-derived iPSCs. FIG. 14E is a bar graph showing C9orf72 intron 1 to exon 2 ratio as measured by RNA-seq in c9ALS patient-derived iPSCs (n=1 healtHY IPSC line, 3 replicates per line). FIG. 14F is a plot showing log₂(Fold Change) vs Gene Abundance as measured by RNA-seq in healthy donor-derived iPSCs. The red dots indicate genes significantly up or down regulated transcriptome wide. The blue dot represented C9orf72.

FIGS. 15A-15B show a plot and bar graph showing that ALSFI (labeled as 2) has no effect on healthy C9ORF72 protein levels. Error bars indicate SD. FIG. 15 A shows a Representative Western blot showing total C9ORF72 protein levels in c9ALS iPSCs treated with ALSFI. FIG. 15 B shows a bar graph representing quantification of total C9ORF72 protein levels normalized to β-actin (n=1 C9orf72 IPSC line, 3 replicates per line).

FIGS. 16A-16D present bar graphs showing that ALSFI (labeled as 2) has no effect of iPSCs lacking the r(G₄C₂)^{exp}. Vehicle indicates 0.1% (v/v) DMSO. Error bars indicate SD. FIG. 16A is a bar graph showing that ALSFI has no effect on C9orf72 exon 1b abundance in differentiated c9ALS motor neurons, as measured by rt-QPCR (n=1 C9orf72 motor neuron line, 3 replicates each). FIG. 16B is a bar graph showing that ALSFI has no effect on C9orf72 intron 1 abundance in iPSNs differentiated from healthy donor iPSCs, as measured by rt-QPCR (n=1 motor neuron lines, 3 replicates per line). FIG. 16C a bar graph showing that ALSFI has no effect on C9orf72 exon 2-3 abundance in iPSNs differentiated from healthy donor iPSCs, as measured by rt-QPCR (n=1 motor neuron lines, 3 replicates per line). FIG. 16D is a bar graph showing that ALSFI has no effect on C9orf72 exon 1b abundance in iPSNs differentiated from healthy donor iPSCs, as measured by rt-QPCR (n=1 motor neuron lines, 3 replicates per line).

FIGS. 17A-17L present blots and bar graphs showing the results of targeted siRNA knockdown validation experiments. Vehicle indicates 0.1% (v/v) DMSO. *P<0.05, **P<0.001, ***P<0.0001, ****P<0.0001 as determined by a t-test with Welch's correction (FIGS. 17B, 17E, 17H, and 17K) or a One-Way ANOVA with multiple comparisons (FIGS. 17C, 17F, 17I, & 17L). Error bars indicate SD. FIG. 17A is a Western blot compariNG HRRP6 protein abundance to β-actin protein abundance in C9orf72 iPSCs treated with vehicle or AN HRRP6-targeting siRNA. FIG. 17B is a bar graph showing quantification of western blot in FIG. 17A (n=1 C9orf72 IPSC line, 3 replicates per line). FIG. 17C is a bar graphs showing the validation OF HRRP6 transcript knockdown upON HRRP6-targeting siRNA treatment in C9orf72 iPSCs, as measured by rt-QPCR (n=1 C9orf72 ISPC line, 5 replicates per line). FIG. 17D shows a Western blot comparing RNase L protein abundance to β-tubulin protein abundance in C9orf72 iPSCs treated with vehicle or a RNase L-targeting siRNA. FIG. 17E is a bar graph showing the quantification of western blot in FIG. 17D. FIG. 17F is a bar graphs showing the validation of RNase L transcript knockdown upon RNase L-targeting siRNA treatment in C9orf72 iPSCs, as measured by rt-QPCR. FIG. 17G shows a Western blot comparing XRN1 protein abundance to vinculin protein abundance in C9orf72 iPSCs treated with vehicle or a XRN1-targeting siRNA. FIG. 17H is a bar graph showing the quantification of western blot in FIG. 17G. FIG. 17I is a bar graph showing the validation of XRN1 transcript knockdown upon XRN1-targeting siRNA treatment in C9orf72 iPSCs, as measured by rt-QPCR. FIG. 17J shows a Western blot comparing XRN2 protein abundance to β-tubulin protein abundance in C9orf72 iPSCs treated with vehicle or AN HRRP6-targeting siRNA. FIG. 17K is a bar graph showing the quantification of western blot in FIG. 17J. FIG. 17L is a bar graph showing the validation of XRN2 transcript knockdown upon XRN2-targeting siRNA treatment in C9orf72 iPSCs, as measured by rt-QPCR.

### DETAILED DESCRIPTION

Elimination of r(G₄C₂)^{exp} could possibly ameliorate all c9ALS/FTD molecular defects, a significant advantage over targeting a particular c9ALS/FTD pathway. This strategy warrants benefits especially with r(G₄C₂)^{exp} presence within an intron, and not an open reading frame.

To enable such an approach, a high affinity compound, a pyridocarbazole compound of Formula II was developed to selectively recognize r(G₄C₂)^{exp} through the use of structure-activity relationships (SAR), biophysical, and structural analyses. Research involving the binding compound of Formula II led to the ALS compound of Formula I which exhibits effective reduction, minimization and/or elimination of r(G₄C₂)^{exp} and its downstream disease-associated pathologies.

Rational Design of a Monomeric Compound that Binds r(G₄C₂)^{exp}.

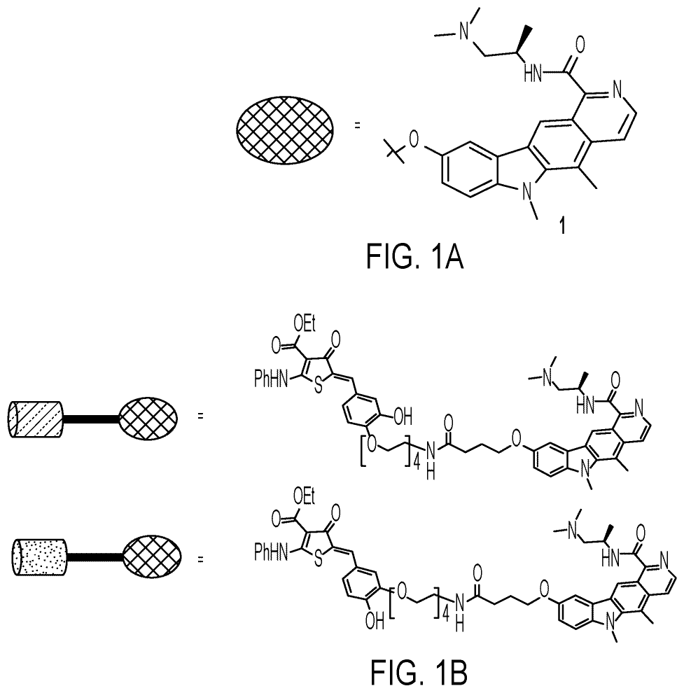

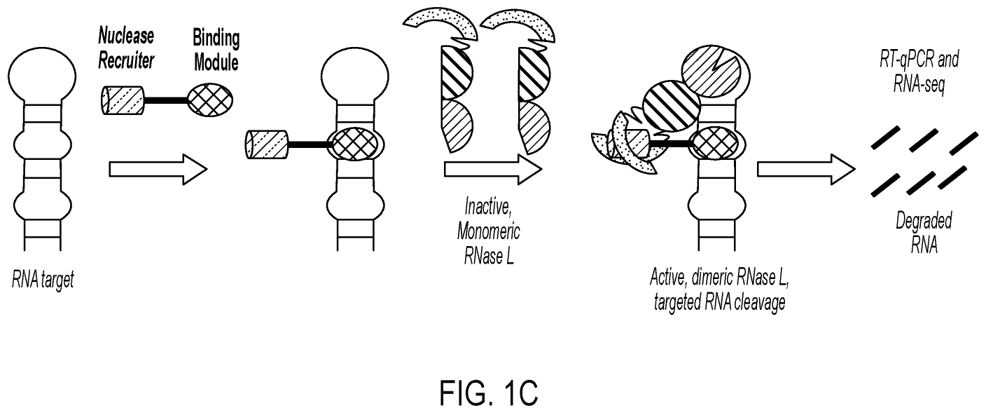

Application of the ReFrame library along with SAR chemical modification coupled with HEK293T cellular screening, led to selection of a r(G₄C₂)^{exp} binding compound of Formula II (labeled as compound 1 in FIG. 1A). Compound Formula II (labeled as 1 in Figures) binds to the r(G₄C₂)^{exp} with a K_{d} of 560±160 nM and reduces poly(GP), the most soluble DPR, abundance and nuclear foci formation in patient-derived cellular systems. Additionally, binding of the Compound of Formula II to the hairpin structure of the r(G₄C₂)^{exp} results in the endogenous decay of C9orf72 intron 1, thus eliminating the disease-causing RNA through native RNA quality control pathways.

To prolong the effectiveness of Compound Formula II in cells and to enable effective degradation and/or reduction of repeat translation of r(G₄C₂)^{exp}, Compound Formula II was converted into a ribonuclease targeting chimera, ALS Compound Formula I (labeled as 2; FIG. 1B). This ALS Compound Formula I consists of Compound Formula II linked to a ribonuclease L (RNase L) recruiting moiety of Formula IIIA via a short polyethylene glycol (PEG) linker of Formula IV (See also FIG. 1). RNase L is an endogenous ribonuclease that is present in small quantities as an inactive monomer ubiquitously in cells.^{23,24} During an immune response to a viral infection the cell synthesizes 2′-5′ polyadenylate (polyA), the endogenous ligand of RNase L, to dimerize and active the ribonuclease, thus inducing cleavage of the invading viral RNA.^{23,25} However, in the case of RIBOTAC-type compounds, e.g., compounds bearing the RNase L recruiting moiety of Formula IIIA, the RNase L recruiting moiety Formula IIIA locally activates RNase L in close proximity to a disease-causing RNA transcript, thus resulting in selective cleavage of the target RNA, without eliciting a wide-spread immune response (FIGS. 1C & 7).²⁶⁻²⁹

### ALS Compound Formula I Inhibits RAN Translation in a Transfected Cellular Model.

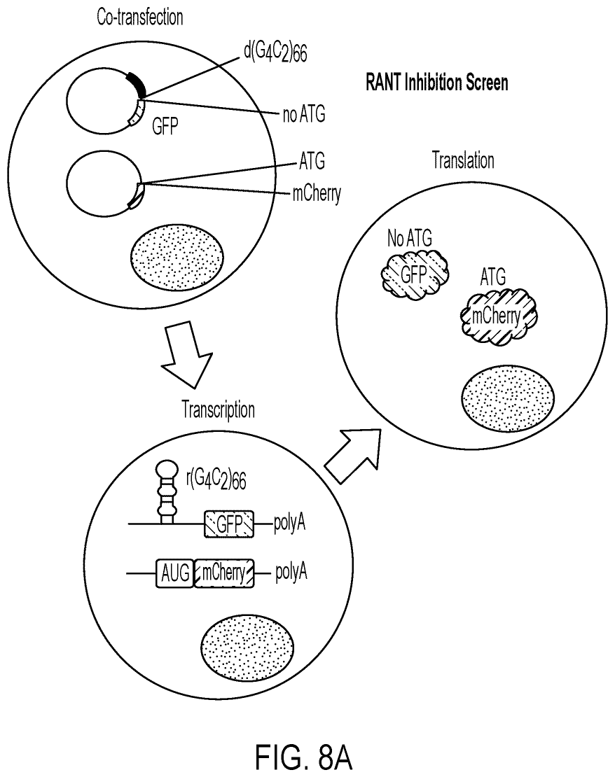

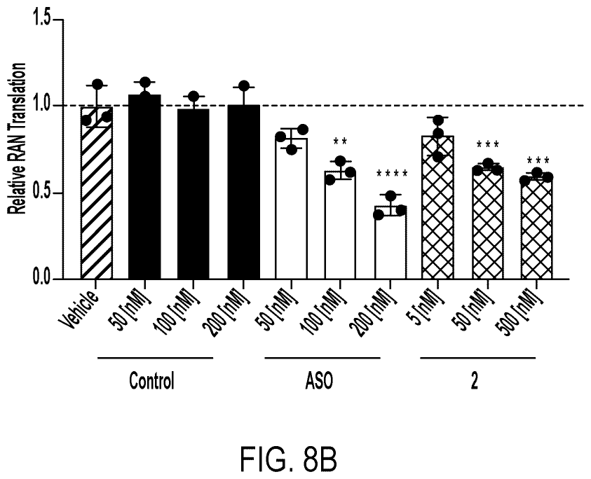

ALS Compound Formula I was tested in the RAN translation assay in HEK293T cells to assess whether addition of the RNase L recruiting moiety Formula IIIA affected the small molecule's ability to reduce RAN translation (FIG. 8A). ALS Compound Formula I reduced the GFP signal in a dose-dependent manner with an IC₅₀ of ˜500 nM (FIG. 8B), indicating the attached RNase L recruiting moiety, Formula IIIA, does not interfere with ALS Compound Formula I's ability to reduce RAN translation. ALS Compound Formula I had no effect on the mCherry signal, indicating canonical translation is not affected by the RIBOTAC.

### Affinity and Selectivity of ALS Compound Formula I.

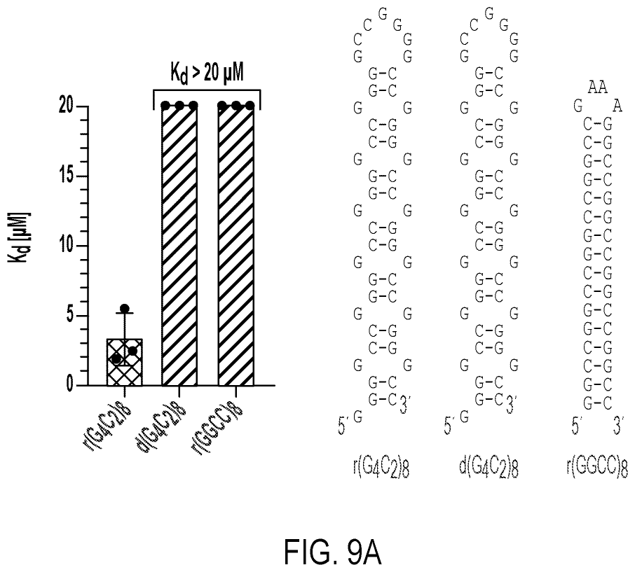

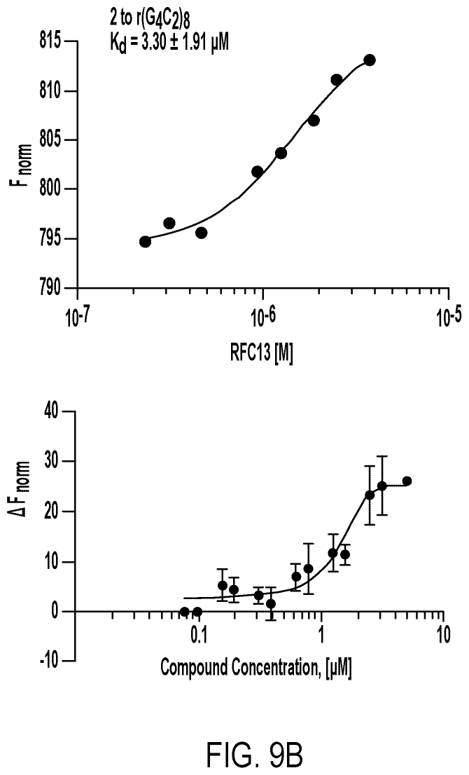

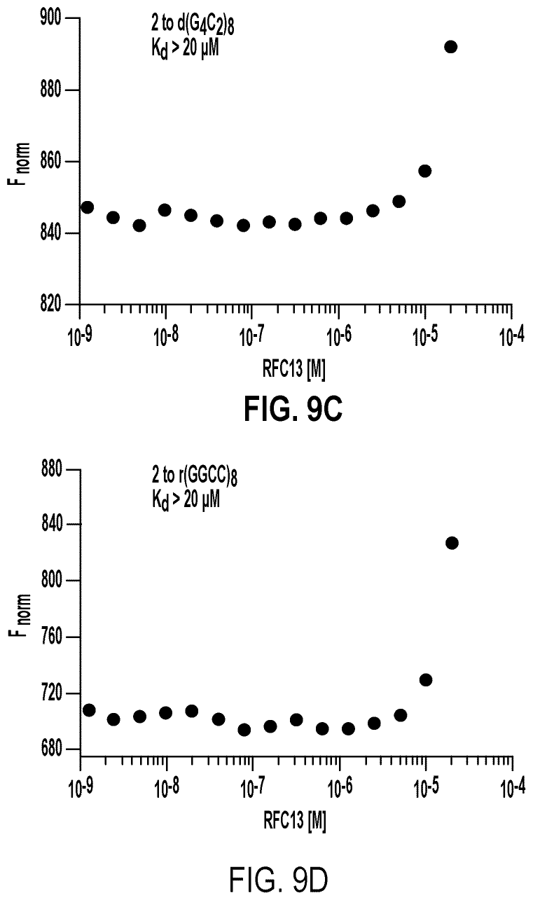

The in vitro affinity and selectivity of ALS Compound Formula I was then measured by microscale thermophoresis (MST). Three nucleic acid constructs were used to assess small molecule binding. ALS Compound Formula I bound to r(G₄C₂)₈ (SEQ ID NO: 1), a mimic of the r(G₄C₂)^{exp}, with a K_{d}=3.30±1.91 μM (FIGS. 9A-9B). No saturable binding was observed to a base-paired control construct, r(GGCC)₈ (SEQ ID NO: 47), or d(G4C₂)₈ (SEQ ID NO: 1), at concentrations up to 20 μM, indicating ALS Compound Formula I is selective for the structured hairpin of r(G₄C₂)₈ (SEQ ID NO: 1), which is lacking in the base-paired control and DNA construct (FIGS. 9A, 9C, & 9D).

In Vitro Validation of RNase L-Mediated Cleavage of the r(G₄C₂)^{exp} by ALS Compound Formula I.

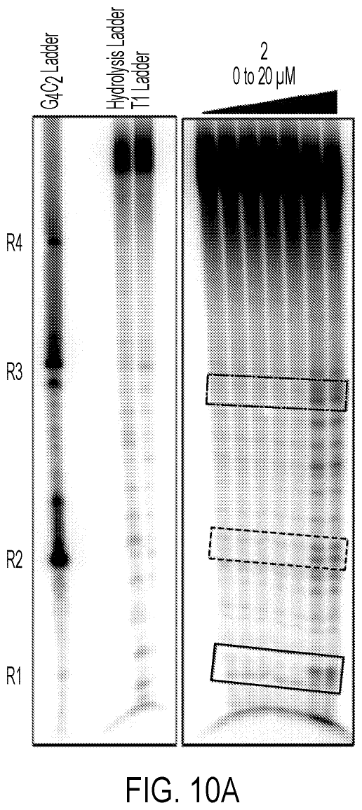

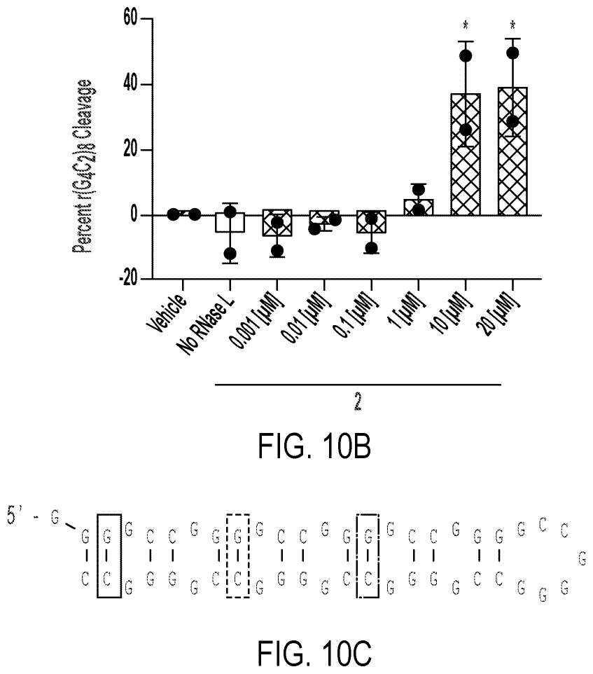

The ability of ALS Compound Formula I to cleave r(G₄C₂)₈ (SEQ ID NO: 1) in vitro, via RNase L recruitment was assessed. ³²P radiolabeled r(G₄C₂)₈ (SEQ ID NO: 1) was treated with a constant concentration of RNase L and increasing concentrations of ALS Compound Formula I, and the resulting fragments analyzed by polyacrylamide gel electrophoresis. At doses of 10 μM and 20 μM of ALS Compound Formula I, significant cleavage of r(G₄C₂)₈ (SEQ ID NO: 1) was observed, indicating RNase L is recruited by ALS Compound Formula I in vitro to cleave the target RNA. Cleavage sites were mapped to the GC base pairs proceeding each 1×1 GG internal loop in the hairpin structure of r(G₄C₂)₈ (SEQ ID NO: 1) (FIG. 10).

### Cellular Toxicity of ALS Compound Formula I.

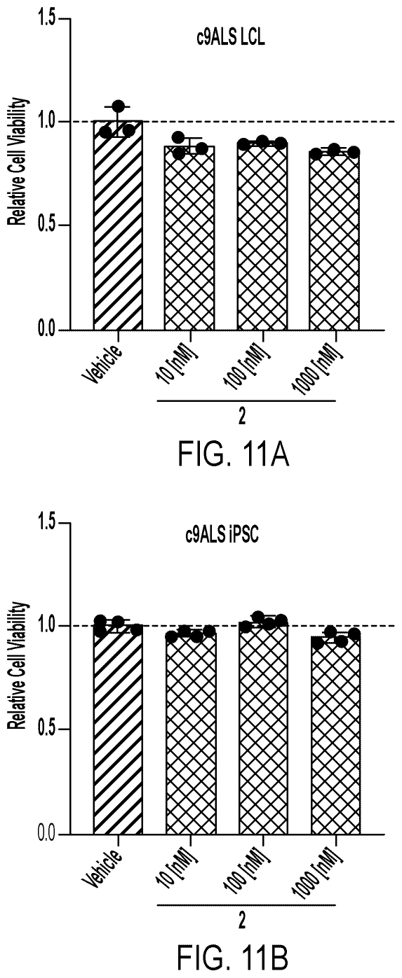

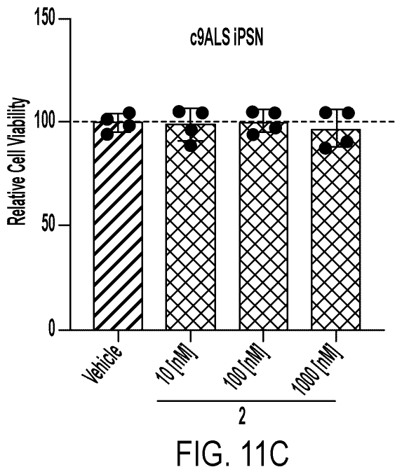

To assess ALS Compound Formula I's ability to rescue c9ALS/FTD-associated pathologies in cells, we utilized three types of cell lines from both c9ALS/FTD patients and healthy donors; 1) lymphoblastoid cell lines (LCLs); 2) induced pluripotent stem cells (iPSCs); and 3) iPSC differentiated motor neurons (iPSNs). In all three cell lines ALS Compound Formula I showed no significant toxicity at doses up to 1 μM (FIG. 11).

ALS Compound Formula I Engages r(G₄C₂)^{exp} in Patient-Derived Cellular Models.

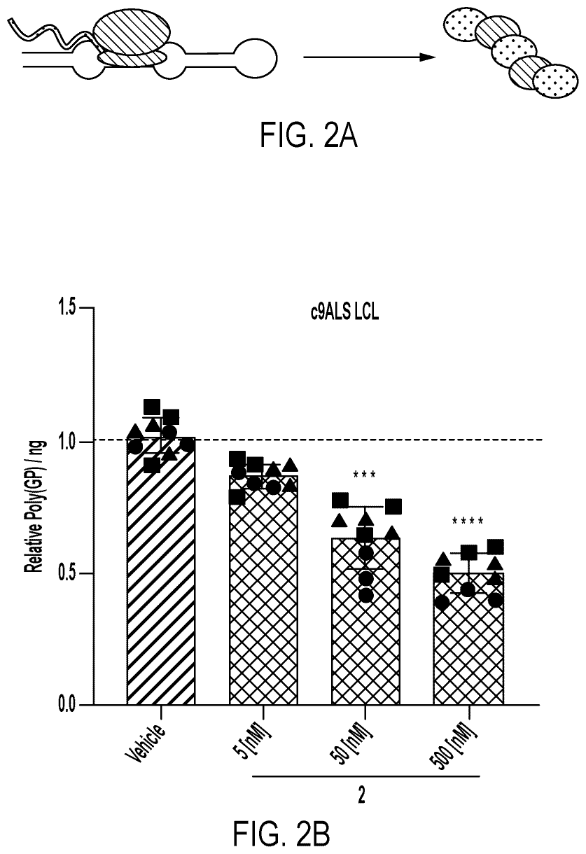

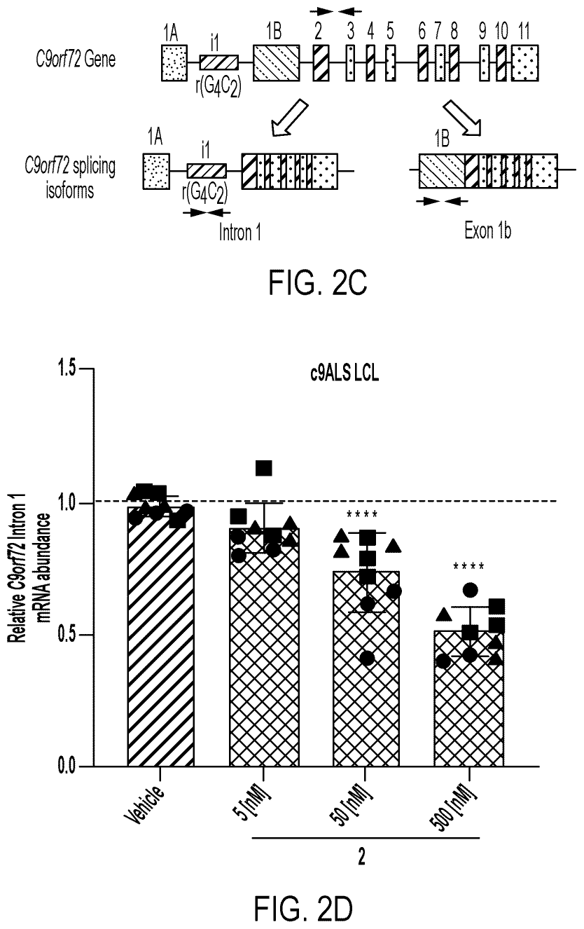

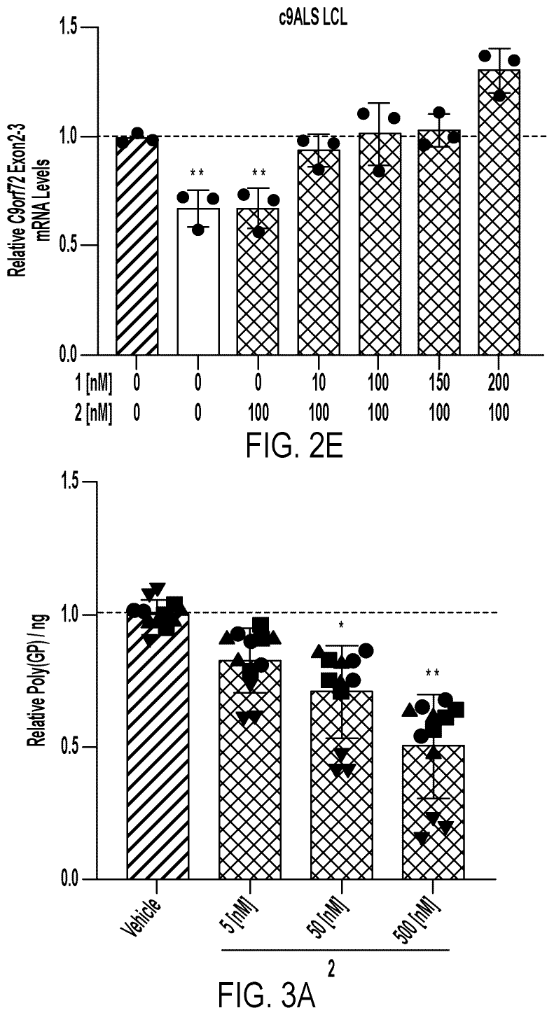

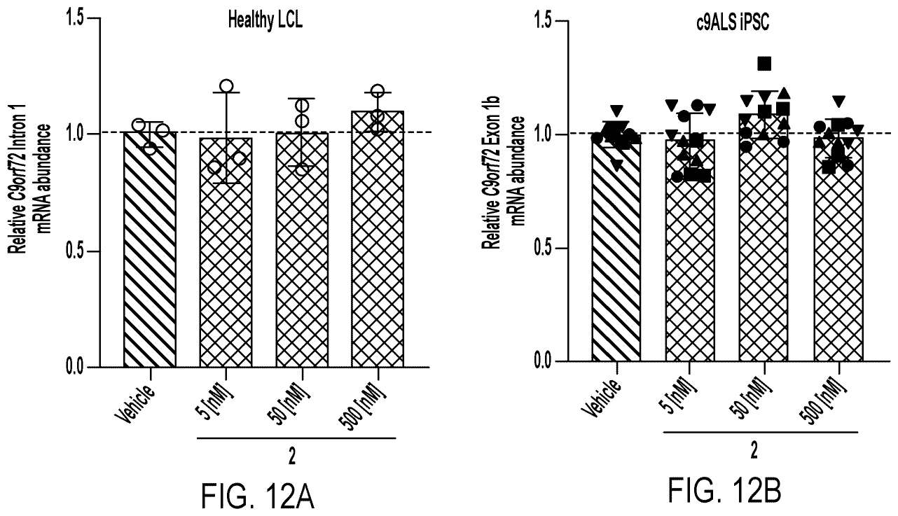

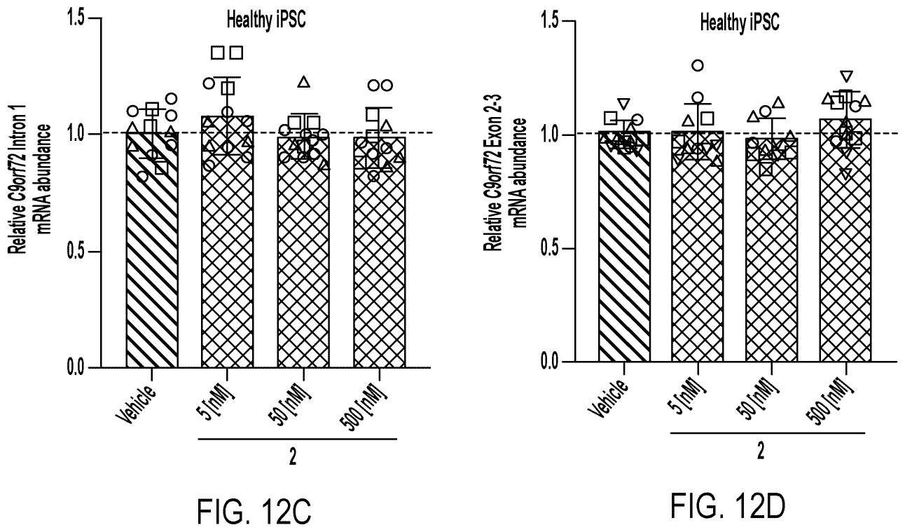

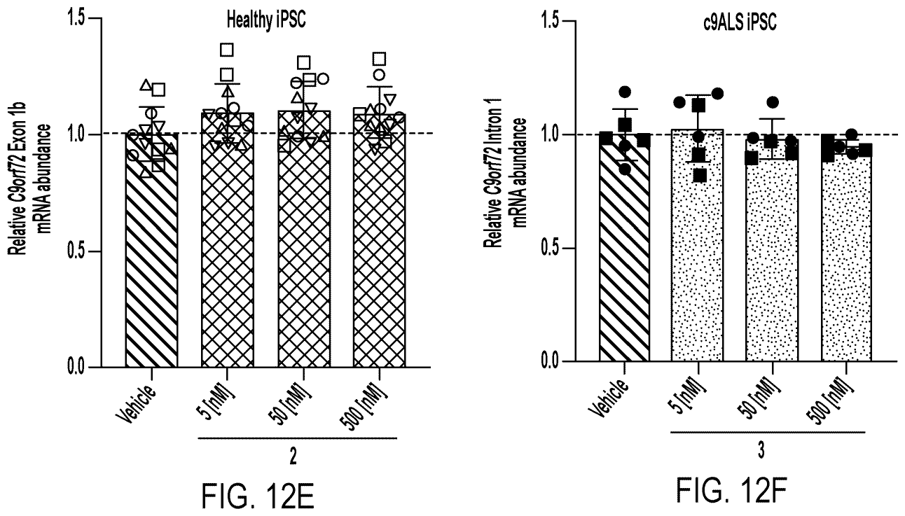

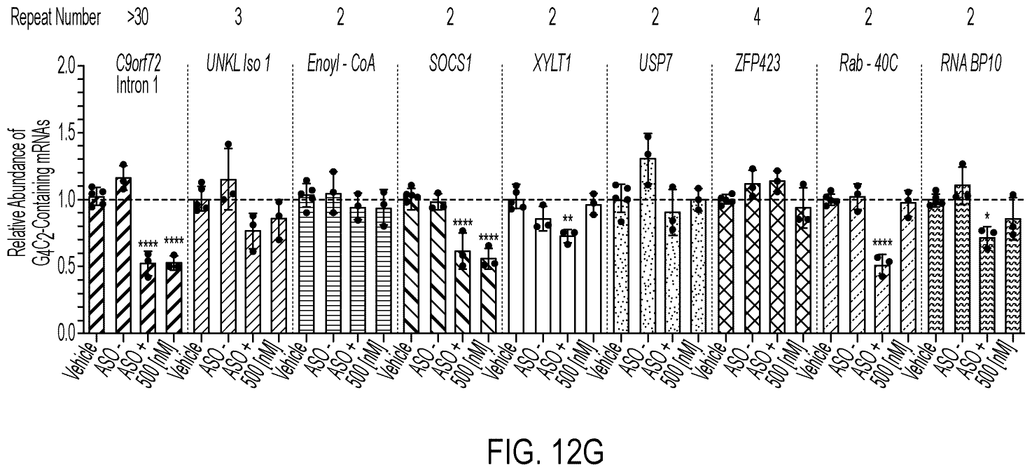

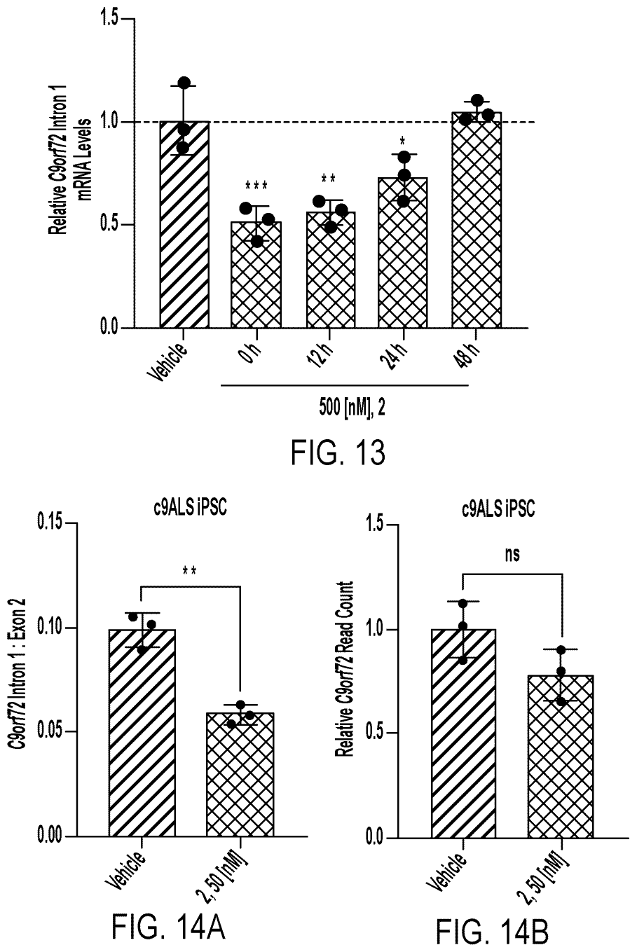

ALS Compound Formula I was first tested in c9ALS/FTD patient-derived LCLs to test its ability to reduce c9ALS/FTD-associated cellular pathologies. ALS Compound Formula I reduced RAN translation, as measured by poly(GP) abundance, by ˜50% at a dose of 500 nM, using an electroluminescent sandwich immunoassay as a read out (FIGS. 2A-2B). Additionally, real time quantitative polymerase chain reaction (RT-qPCR) was used to measure the abundance of three C9orf72 transcript isoforms. Primers were designed specific for either: 1) the r(G₄C₂°)^{exp}-containing intron 1 of C9orf72; 2) exon 1b of C9orf72, which is only present in properly spliced isoforms that do not contain the r(G₄C₂)^{exp}; or 3) the exon 2-exon 3 junction which is present in all C9orf72 isoforms (i.e., those both including and excluding the r(G₄C₂)^{exp}) (FIG. 2C). Using primers specific for intron 1 of C9orf72 revealed an ˜50% reduction in intron 1 abundance when ALS Compound Formula I was treated at 500 nM (FIG. 2D). However, in lymphoblastoid cells derived from healthy donors, treatment with ALS Compound Formula I did not reduce C9orf72 intron 1 levels, as measured by RT-qPCR (FIG. 12A). Poly(GP) was undetectable in cells from healthy donors by our electroluminescent sandwich immunoassay, as expected for cells that do not harbor the r(G₄C₂)^{exp} in intron 1 of C9orf72.

A competition assay was conducted between Compound Formula II and ALS Compound Formula I to confirm target engagement of ALS Compound Formula I with r(G₄C₂)^{exp}. However, since treatment with Compound Formula II also causes a reduction in C9orf72 intron 1 levels, RT-qPCR was completed using primers spanning the exon 2-exon 3 junction of C9orf72 (as a measure of the full C9orf72 transcript, not just the intron containing the RNA repeat). Cells were treated with increasing doses of Compound Formula II and a constant concentration of ALS Compound Formula I (100 nM). As expected, increasing concentrations of Compound Formula II competed with ALS Compound Formula I for binding of the lxi GG internal loops, thus decreasing the cleavage observed by RT-qPCR when exon 2-3 primers were used (FIG. 2E). These data confirm Compound Formula II and ALS Compound Formula I both target the r(G₄C₂)^{exp} repeat in cells.

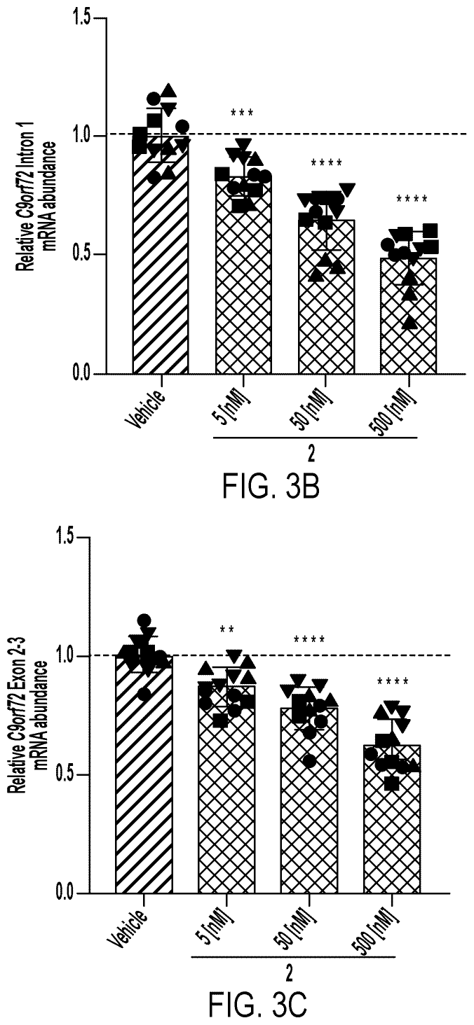

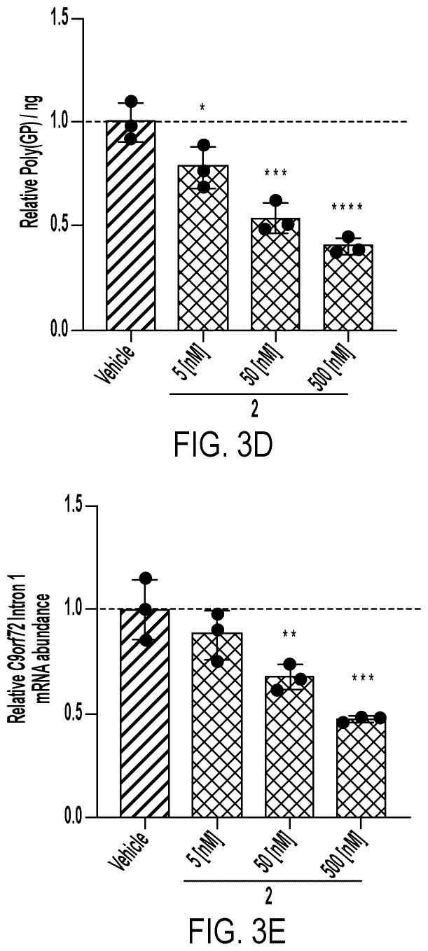

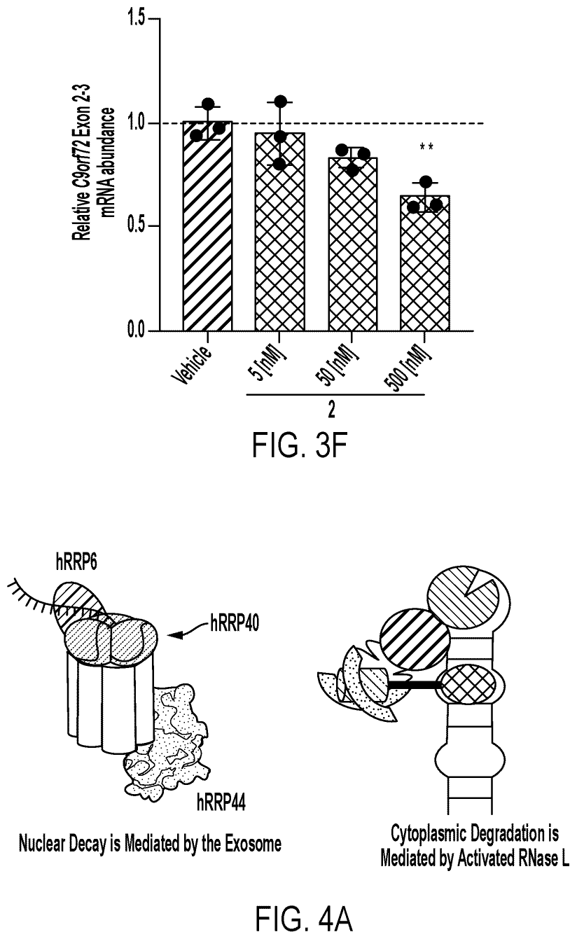

A more advanced cellular model of c9ALS/FTD, patient-derived iPSCs, was also examined. The effect of ALS Compound Formula I on poly(GP) and C9orf72 intron 1 abundance was measured. ALS Compound Formula I decreased poly(GP) abundance by ˜50% at 500 nM and remained decreased by ˜20% 48 hours after treatment (FIG. 3D). Interestingly, 48 hours after treatment with Compound Formula II, poly(GP) abundance is restored to that as vehicle-treated samples (FIG. 3). C9orf72 intron 1 abundance was also reduced by ˜50% upon treatment with 500 nM of ALS Compound Formula I (labeled as 2, FIG. 3B). C9orf72 exon 2-3 abundance was reduced by only ˜30%, as measured by RT-qPCR, consistent with the fact that the exon 2-3 junction is present in both the r(G₄C₂)^{exp}-containing exon 1a isoform and the non-repeat containing, alternatively spliced exon 1b isoform, thus exon 2-3 primers measure both the abundance of healthy and disease-causing C9orf72 transcripts (FIG. 3C). Additionally, primers specific for the exon 1b isoform, an exon only present in properly spliced healthy C9orf72 transcripts, showed no decrease as measured by RT-qPCR upon treatment with ALS Compound Formula I, further confirming ALS Compound Formula I's selectivity for the disease-causing transcript (FIG. 12B). IPSCs derived from healthy donors showed no decrease in C9orf72 intron 1, exon 2-3 or exon 1b levels, as measured by RT-qPCR using the appropriate primers (FIGS. 12C-12E). This is expected as healthy cells do not contain the structured r(G₄C₂)^{exp}. It is significant that ALS Compound Formula I carrying the ribonuclease recruitment module Formula IIIA allows for the complete cleavage of the disease-associated isoform, as assessed by exon 2-3 abundance. This cleavage resulted in a prolonged bioactivity as measured by a washout experiment showing that, upon removal of the compound from cell culture medium, it takes up to 48 hours for the abundance of C9orf72 intron 1 to return to levels before compound intervention (FIG. 3).

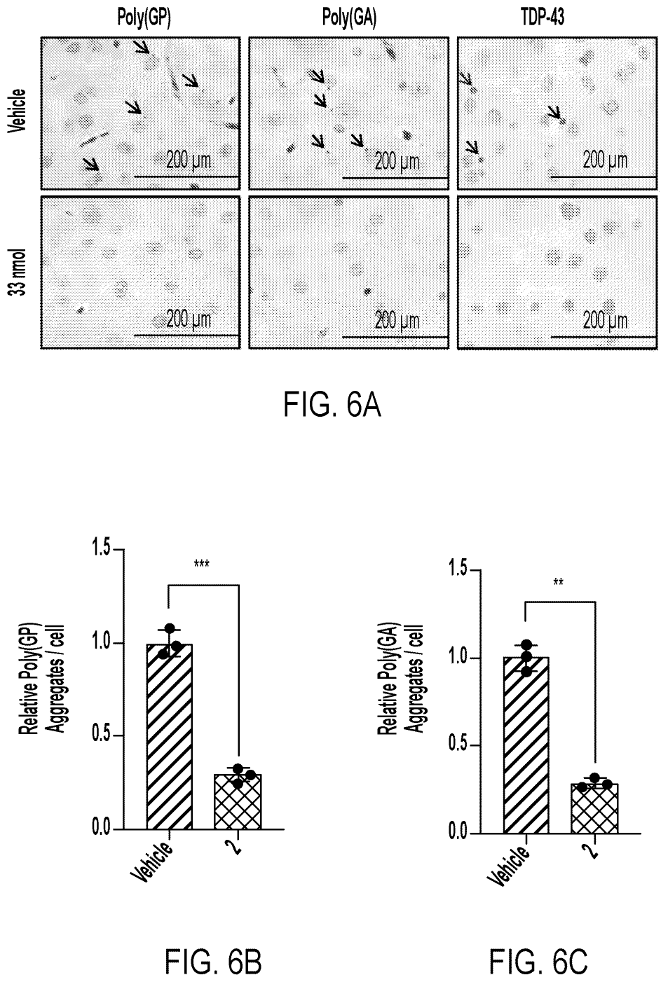

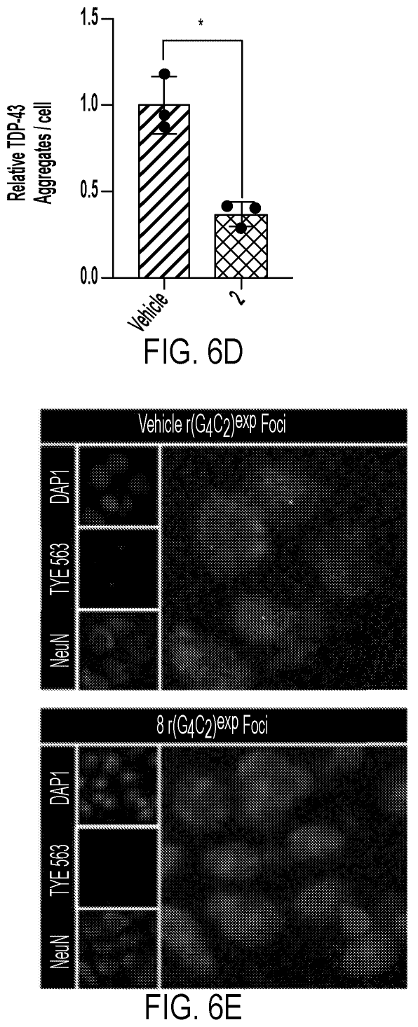

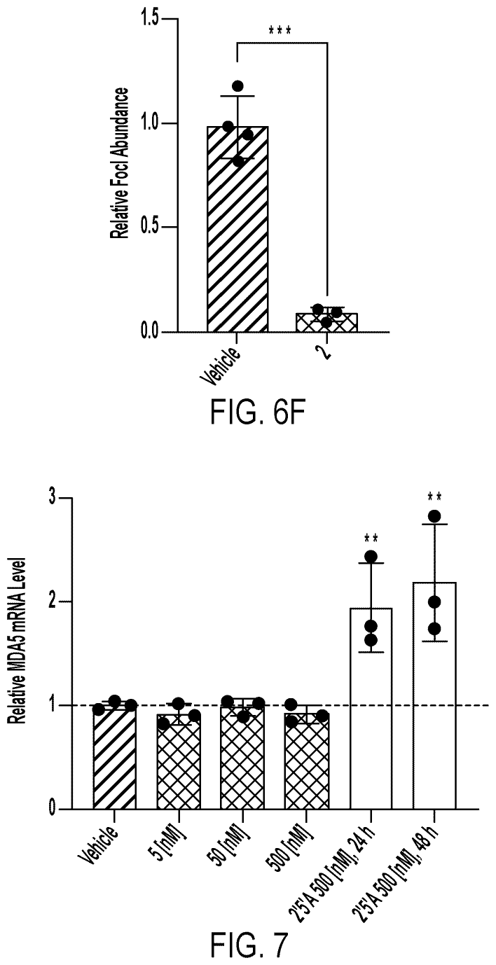

Additionally, a less efficient RIBOTAC of Compound Formula II (Compound 3, FIG. 1B) was synthesized, through use of a link with a stereoisomer of the RNase L recruiting module that is significantly less effective of Formula IIIA, R″ being the link and R′ being H (Compound 3, FIG. 1B). Thus, Compound 3 lacks the ability to recruit RNase L and cleave the full C9orf72 transcript, but still acts through a simple binding mechanism of action like Compound Formula II. (FIG. 6F).

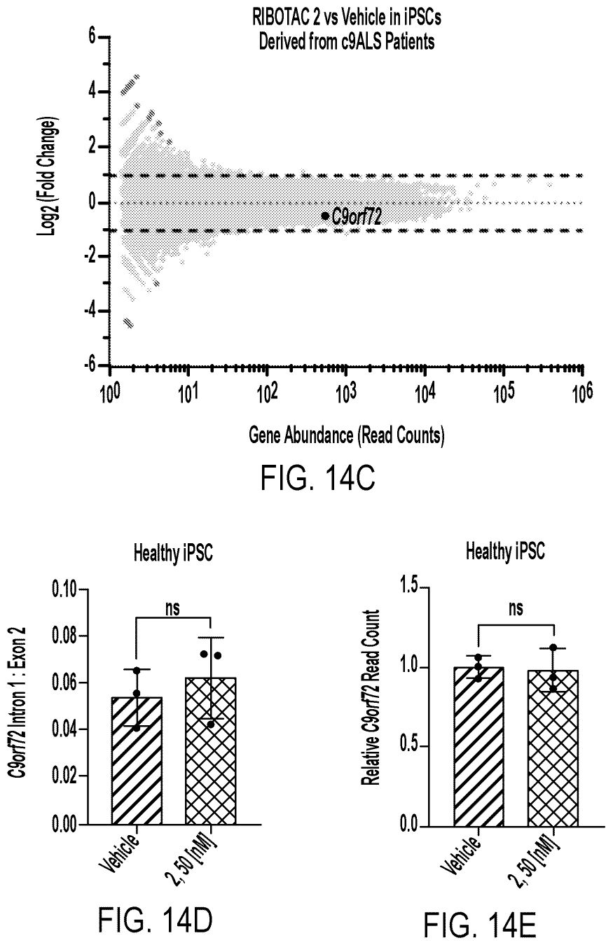

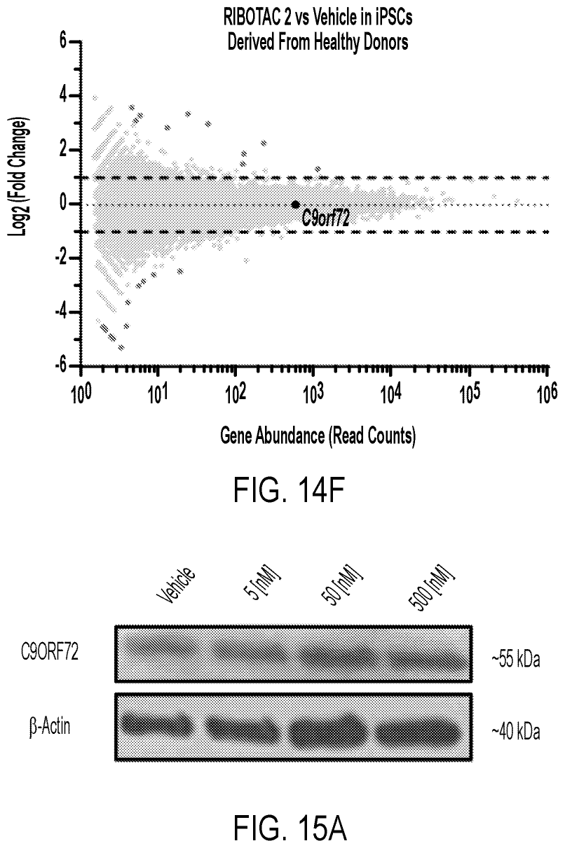

Furthermore, using RT-qPCR to measure the abundance of eight additional transcripts that contain shorter, non-pathogenic r(G₄C₂) (SEQ ID NO: 48) repeats (repeat length 2-4, i.e., do not form an RNA hairpin structure) showed only one transcript, SOCS1, was significantly reduced upon treatment with ALS Compound Formula I, while treatment with a r(G₄C₂)^{exp}-targeting ASO significantly decreased the levels of four of the transcripts (FIG. 12G). However, SOCS1 was not found to be significantly down-regulated when RNA-sequencing (RNA-seq) was performed (FIG. 14C). These data further support that ALS Compound Formula I recognizes the structure of the r(G₄C₂)^{exp}, not the primary sequence, making the small molecule more selective than ASO's for targeting this RNA repeat.

Additionally, RNA-seq analysis of c9ALS/FTD patient-derived iPSCs showed that treatment with ALS Compound Formula I (50 nM) significantly reduced the intron 1:exon 2 ratio, while having no effect on the total C9orf72 read counts, and minimal off-targets transcriptome-wide (FIGS. 14A-14C). In iPSCs derived from healthy donors, ALS Compound Formula I elicited no effect on the intron 1:exon 2 ration or C9orf72 read count and had minimal off-target effects transcriptome-wide (FIGS. 14D-14F).

The effect of ALS Compound Formula I on total C9ORF72 protein levels in c9ALS/FTD patient-derived iPSCs was investigated. Treatment with ALS Compound Formula I had no effect on healthy C9ORF72 protein levels, as expected, considering the C9ORF72 protein is translated from properly spliced C9orf72 transcripts that do not contain the r(G₄C₂)^{exp} (FIGS. 15A-15B) Thus, reducing the abundance of r(G₄C₂)^{exp}-containing transcripts will not affect the levels of healthy C9orf72 transcripts present.

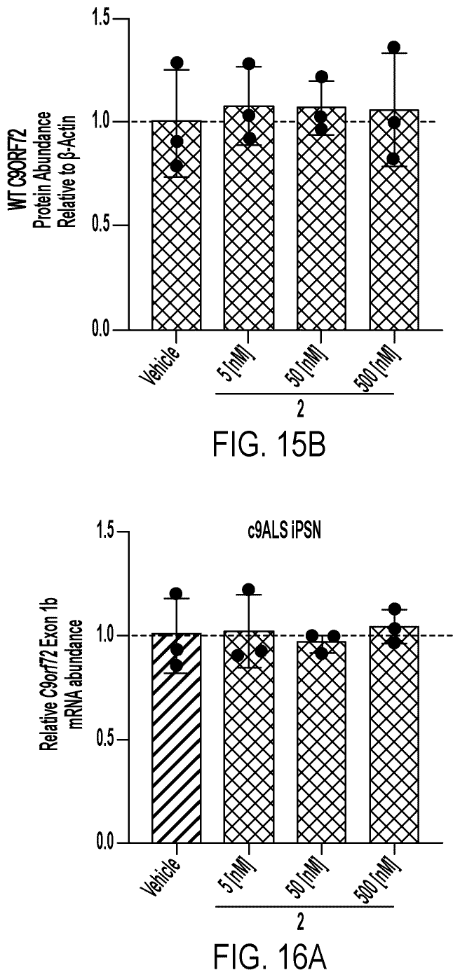

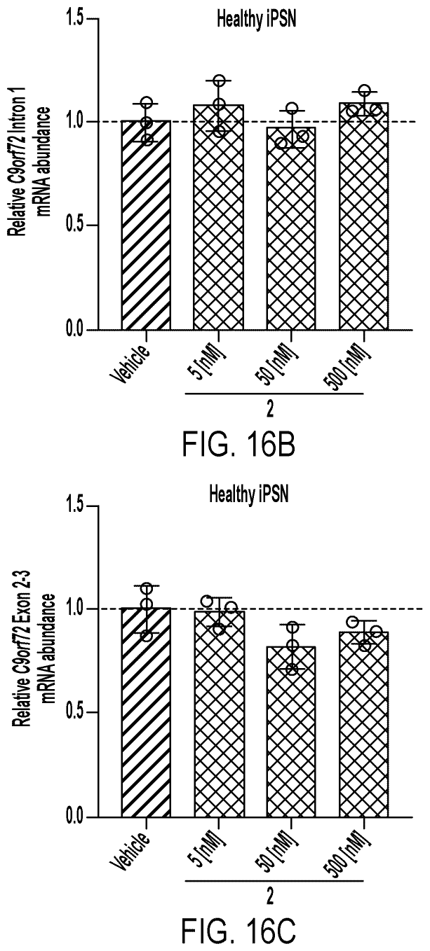

The c9ALS/FTD iPSCs were differentiated into spinal neurons (iPSNs), following a detailed 32-day differentiation protocol previously described. At the end of 32 days, RNA and protein were harvested from the iPSNs and analyzed as described above. When ALS Compound Formula I was treated over the course of 17 days (from day 15-32 of differentiation) poly(GP) abundance was reduced by ˜60% at 500 nM in c9ALS/FTD iPSNs (FIG. 3E). Additionally, RT-qPCR analysis showed a dose-dependent reduction in C9orf72 intron 1 levels (˜50% decrease at 500 nM of ALS Compound Formula I; FIG. 3F) and exon 2-3 abundance (˜40% decrease at 500 nM of ALS Compound Formula I; FIG. 3G), while exon 1b transcript abundance was unaffected (FIG. 16A). No effect was observed on intron 1, exon 2-3 or intron 1b levels in healthy iPSNs upon compound treatment (FIGS. 16B-16D). These data indicate that ALS Compound Formula I has biologically relevant activity in complex cellular models of c9ALS/FTD.

### ALS Compound Formula I Functions Through a Unique Mechanism of Action to Reduce C9orf72 Transcript Abundance.

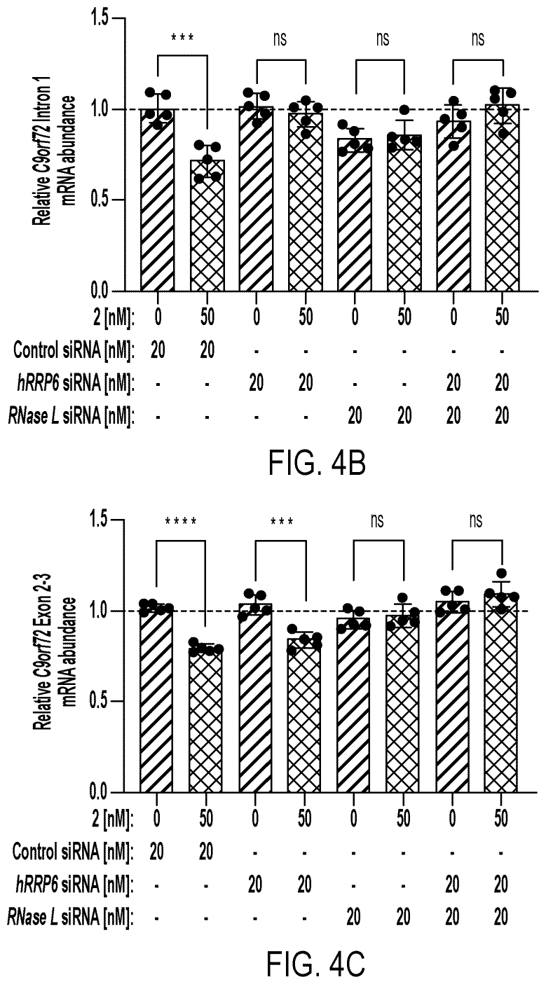

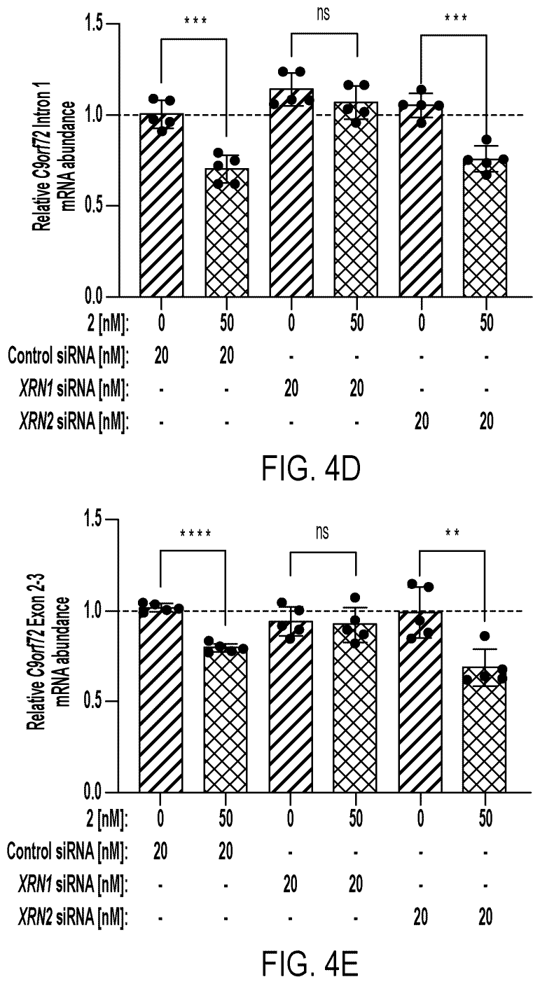

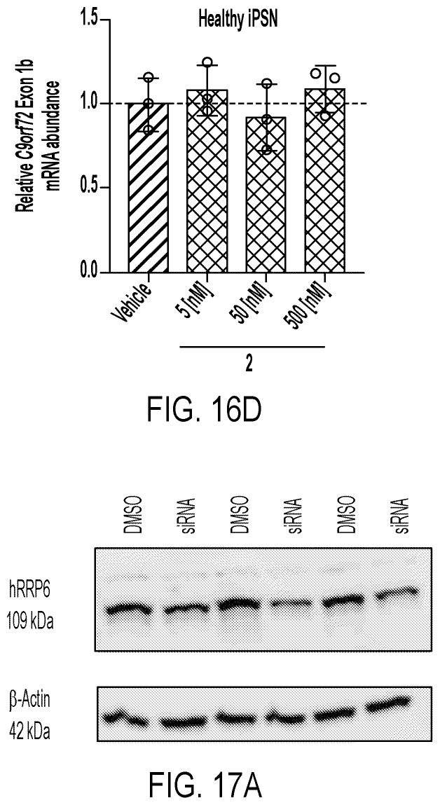

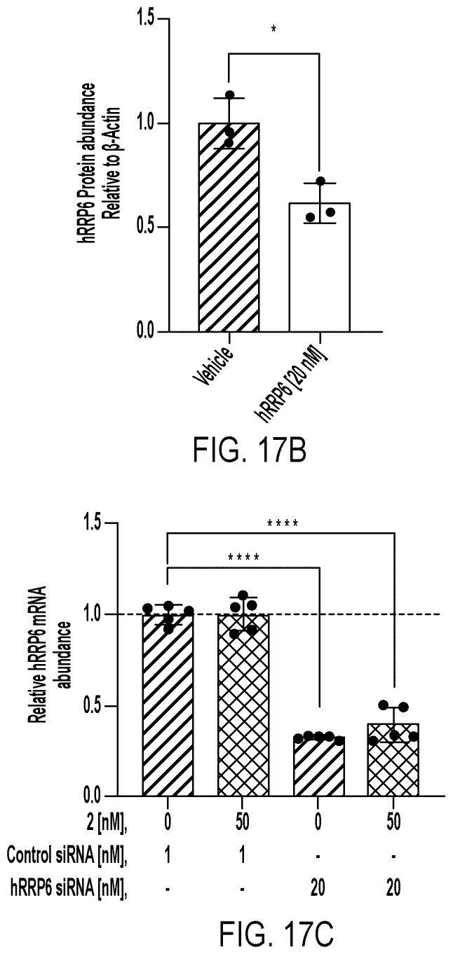

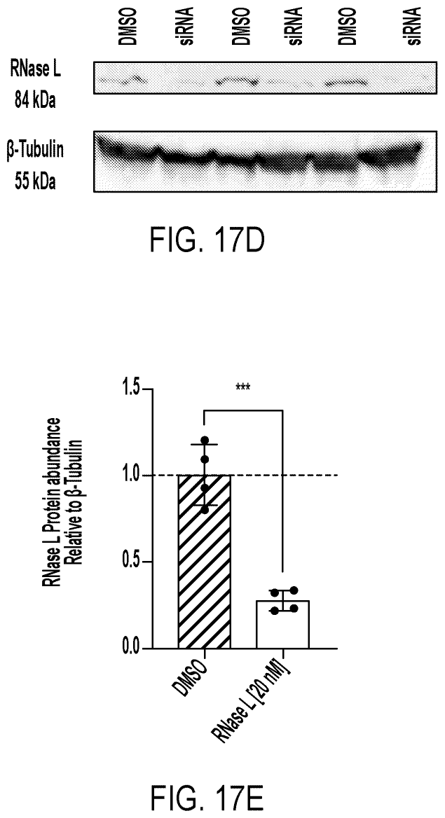

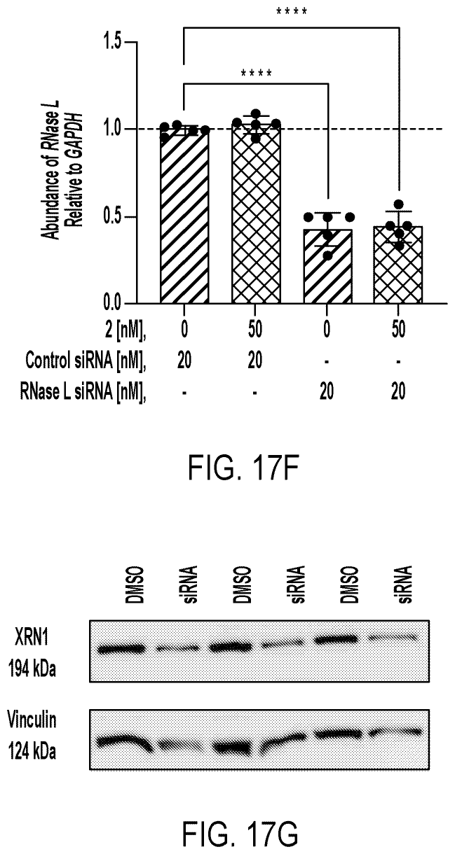

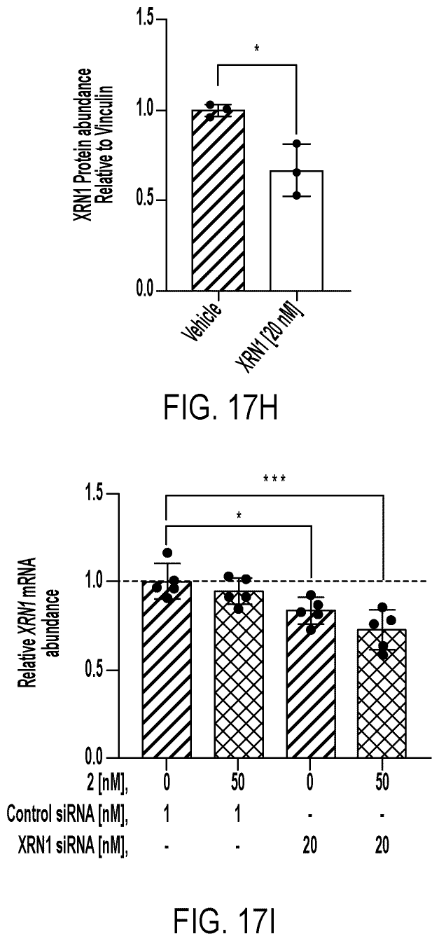

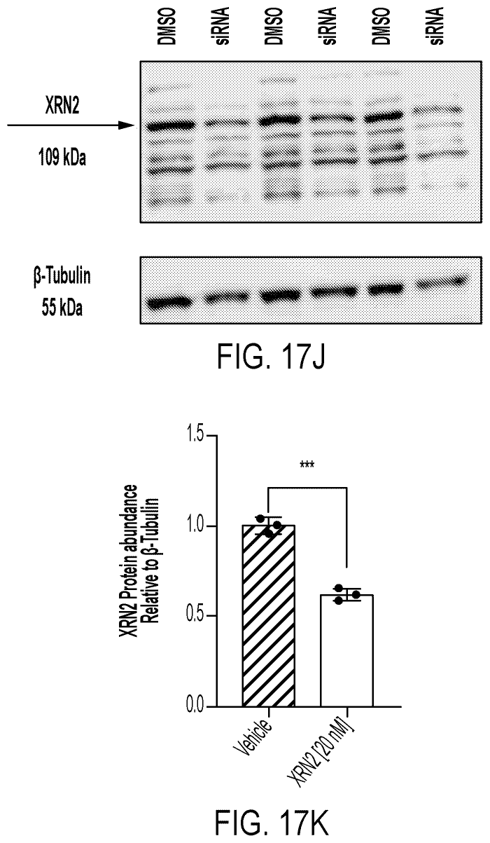

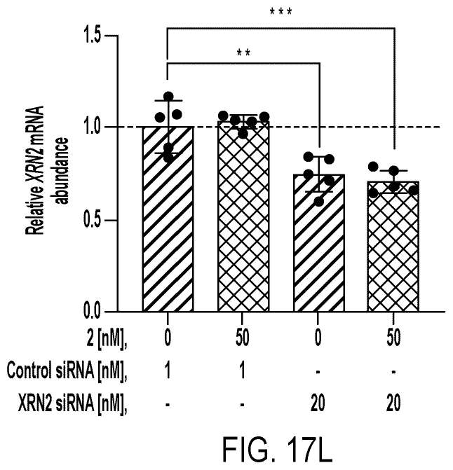

Previous studies with the parent RNA binder, Compound Formula II, demonstrated that the small molecule has a unique ability to induce C9orf72 intron 1 decay without degrading the entire transcript, that is, the intron harboring the r(G₄C₂)^{exp} is spliced from the pre-mRNA upon Compound Formula II binding while the mature transcript remains intact. Since ALS Compound Formula I shares the same RNA-binding module as Compound Formula II, the mechanism of ALS Compound Formula I-mediated C9orf72 degradation in c9ALS/FTD iPSCs was investigated to see if it was more complex than the RNase L-mediated mechanism of action which is normally characteristic of RIBOTACs (FIG. 4A). Indeed, experiments using siRNAs to individually knock down either RNase L or hRRP6 (one of the catalytic subunits of the exosome which has been shown to play a role in Compound II-mediated intron 1 decay) and co-treatment with ALS Compound Formula I, elucidated a complicated mechanism of degradation in which RNase L-mediated cleavage is the driving force behind the reduction in exon 2-3 abundance, while the RNA binder portion of ALS Compound Formula I drives intron 1 decay, consistent with their targets subcellular localization (FIGS. 4B-4C; FIGS. 17A-17F). Treatment with 20 nM of a hRRP6-targeting siRNA and 50 nM of ALS Compound Formula I shows ablation of intron 1 decay, but no effect on exon 2-3 cleavage, indicating that hRRP6 is only working to degrade the intron, not the entire C9orf72 transcript (FIGS. 4B-4C). Additionally, treatment with 20 nM of an RNase L-targeting siRNA and 50 nM of ALS Compound Formula I shows an ablation of both intron 1 and exon 2-3 degradation, indicating that the C9orf72 transcript is no longer being cleaved (FIGS. 4B-4C). Both RNase L and hRRP6 were then knocked down by siRNA treatment, affording degradation of the transcript comparable to RNase L knock-down alone (FIGS. 4B-4C). Therefore, these data show that ALS Compound Formula I RIBOTAC is working through two unique mechanisms to degrade the r(G₄C₂)^{exp}-containing C9orf72 transcript, separated by their subcellular location. Intron 1 decay is dominated by the nuclear exosome, while RNase L-mediated degradation dominates in the cytoplasm, consistent with the cellular localization of RNase L.

Interestingly, the mechanism of C9orf72 degradation appears quite complex when also considering the exoribonucleases XRN1 and XRN2. These proteins are responsible for 5′-3′ endogenous RNA decay and differ only in their subcellular localization; XRN1 is cytoplasmic while XRN2 is nuclear (FIGS. 17G-17L). Treatment with 20 nM of a XRN1-targeting siRNA and 50 nM of ALS Compound Formula I shows ablation of intron 1 decay and C9orf72 cleavage, while treatment with 20 nM of a XRN2-targeting siRNA and 50 nM of ALS Compound Formula I has no effect on either (FIGS. 4D & 4E). These data are consistent with RNase L-mediated cleavage of C9orf72 in the cytoplasm followed by its complete degradation by XRN1. These data further highlight the importance of subcellular localization in the ALS Compound Formula I-mediated C9orf72 degradation mechanism of action.

ALS Compound Formula I Mitigates c9ALS/FTD Pathology In Vivo.

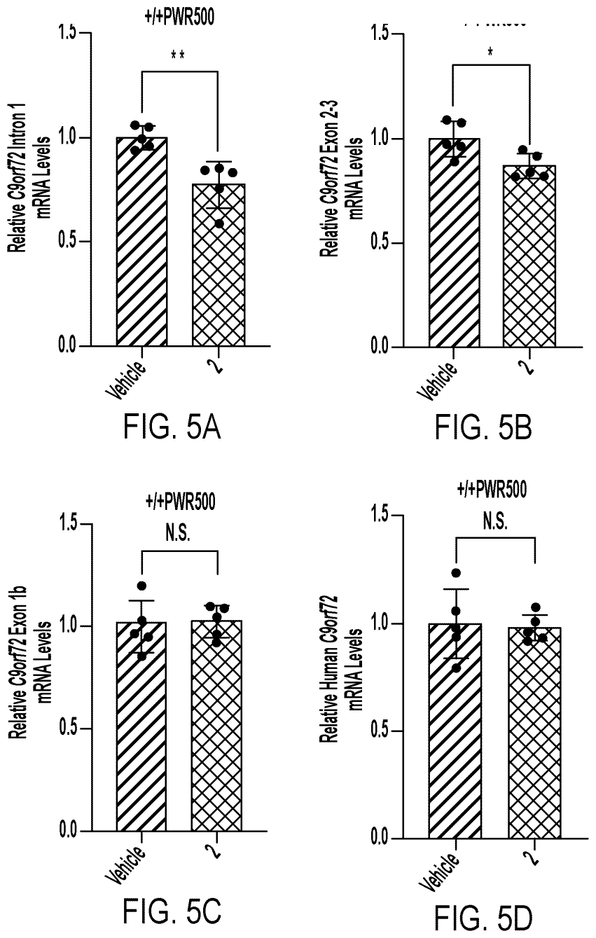

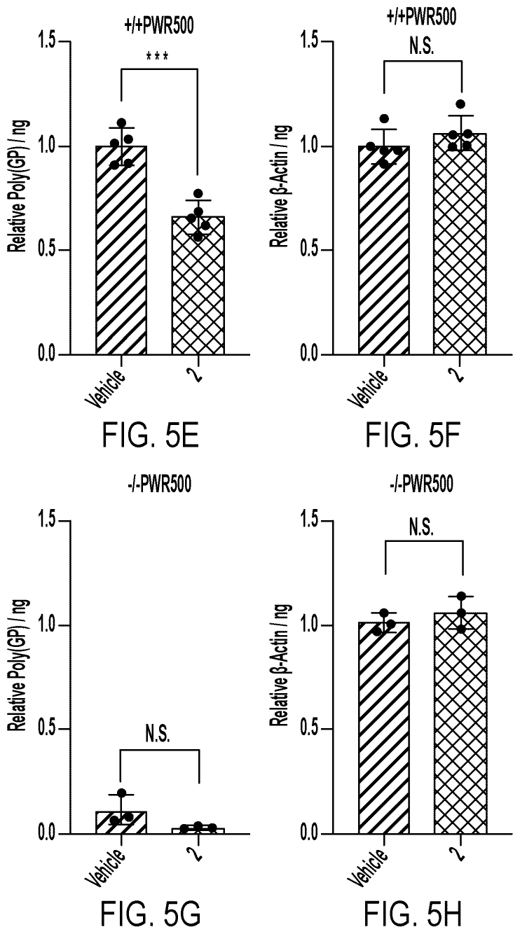

Due to the success of ALS Compound Formula I RIBOTAC in patient-derived cellular models it was next sought to test the ability of the compound to reduce c9ALS/FTD pathologies in vivo. A C9orf72 BAC transgenic (C9BAC) mouse model that expresses ˜500 G₄C₂ repeats (referred to as +/+PWR500 mice) as the model system was utilized. After treatment by a single intracerebroventricular injection (ICV) of 10 mg/kg of ALS Compound Formula I, followed by a 3-week incubation period after which the mice were sacrificed, and brain tissue was harvested for downstream analyses. RT-qPCR of total RNA harvested from total brain tissue showed C9orf72 intron 1 abundance was decreased by ˜25% in +/+PWR500 mice, while exon 2-3 abundance decreased by ˜20% (FIGS. 5A-5B). Exon 1b abundance and primers spanning exon 1b to exon 2 were (represented by human C9orf72 abundance) were unchanged after the treatment course, consistent with the fact that exon 1b is only present in wild-type C9orf72 transcripts lacking the r(G₄C₂)^{exp} (FIGS. 5C-5D).

Protein was also harvested from the brains of these mice and analyzed for poly(GP) abundance. In +/+PWR500 mice, poly(GP) was reduced by ˜40%, while β-actin abundance, used here as a house-keeping protein, was unaffected by treatment with ALS Compound Formula I (FIGS. 5E-5F). Poly(GP) was barely detectable in −/−PWR500 (WT) mice, but β-actin was detected easily and showed no reduction upon treatment with ALS Compound Formula I (FIGS. 5G-5H). Immunohistochemistry (IHC) analysis of cortex slices of +/+PWR500 mice also showed that poly(GP), poly(GA) [another DPR produced from RAN translation] and TDP-43 inclusions [a well-established biomarker for c9ALS/FTD disease progression] are significantly reduced upon treatment with ALS Compound Formula I, further indicating that c9ALS/FTP pathologies can be mitigated by ALS Compound Formula I (FIGS. 6A-6D).

Fluorescence in situ hybridization (FISH) studies, using a TYE563-labeled oligo complementary to the sense strand of the C9orf72 r(G₄C₂)^{exp}, were employed to investigate the effect of ALS Compound Formula I on nuclear foci, another hallmark of c9ALS/FTD, which arises from the sequestration of hnRNP H on the r(G₄C₂)^{exp}. Treatment with ALS Compound Formula I significantly reduced the number of r(G₄C₂)^{exp}-containing foci present in the nuclei of cortex neurons of ALS Compound Formula I-treated +/+PWR500 mice, compared to vehicle treated +/+PWR500 mice, indicating that ALS Compound Formula I alleviates c9ALS/FTD nuclear foci in vivo (FIGS. 6E-6F). Thus, ALS Compound Formula I decreases C9orf72 intron 1 transcript abundance, reduces poly(GP) abundance, disrupts r(G₄C₂)^{exp}-containing nuclear foci, and reduces toxic inclusions in vivo, without causing toxicity to the mouse.

### Mechanism of Action and Medical Treatment

In certain embodiments, the invention is directed to methods of inhibiting, suppressing, depressing and/or managing biolevel translation of the aberrant repeat RNA r(G₄C₂°)^{exp} associated with amyotrophic lateral sclerosis (ALS) and frontotemporal dementia (FTD). These aberrant RNA repeats are present in cell lines, and patients afflicted with ALS and FTD. The ALS Compound can reduce translation of the aberrant repeat RNA by binding the repeats or by inducing cleavage of the repeats. The ALS Compound of Formula I (hereinafter Compounds or Compounds of the invention) as embodiments of the invention for use in the methods disclosed herein bind to the above identified RNA entities and ameliorate and/or inhibit their translation to disease-causing dipeptide repeat proteins as well as formation of foci, nuclear transport.

Embodiments of the Compounds applied in methods of the invention and their pharmaceutical compositions are capable of acting as “inhibitors”, suppressors and or modulators of the above identified RNA entities which means that they are capable of blocking, suppressing or reducing the translation of the RNA entities by simple binding and by facilitating their cleavage.

The Compounds useful for methods of the invention and their pharmaceutical compositions function as therapeutic agents in that they are capable of preventing, ameliorating, modifying and/or affecting a disorder or condition. The characterization of such Compounds as therapeutic agents means that, in a statistical sample, the compounds reduce the occurrence of the disorder or condition in the treated sample relative to an untreated control sample or delays the onset or reduces the severity of one or more symptoms of the disorder or condition relative to the untreated control sample.

The ability to prevent, ameliorate, modify and/or affect in relation to a condition, such as a local recurrence (e.g., pain), a disease known as an ALS/FTD disease may be accomplished according to the embodiments of the methods of the invention and includes administration of a composition as described above which reduces, or delays or inhibits or retards the deleterious medical condition in an ALS/FTD subject relative to a subject which does not receive the composition.

The Compounds of the present invention and their salts and solvates, thereof, may be employed alone or in combination with other therapeutic agents for the treatment of the diseases or conditions associated with the repeat RNA [G₄C₂^{exp}] in intron 1 of chromosome 9 open reading frame 72 (C9orf72).

The Compounds of the invention and their pharmaceutical compositions are capable of functioning prophylactically and/or therapeutically and include administration to the host/patient of one or more of the subject compositions. If it is administered prior to clinical manifestation of the unwanted condition (e.g., disease or other unwanted state of the host animal/patient) then the treatment is prophylactic, (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The Compounds of the invention and their pharmaceutical compositions are capable of prophylactic and/or therapeutic treatments. If the Compounds or pharmaceutical compositions are administered prior to clinical manifestation of the unwanted condition (e.g., disease or other unwanted state of the host animal) then the treatment is prophylactic, (i.e., they protect the host against developing the unwanted condition), whereas if they are administered after manifestation of the unwanted condition, the treatment is therapeutic, (i.e., they are intended to diminish, ameliorate, or stabilize the existing unwanted condition or side effects thereof). As used herein, the term “treating” or “treatment” includes reversing, reducing, or arresting the symptoms, clinical signs, and underlying pathology of a condition in manner to improve or stabilize a subject's condition.

The Compounds of the invention and their pharmaceutical compositions can be administered in “therapeutically effective amounts” with respect to the subject method of treatment. The therapeutically effective amount is an amount of the compound(s) in a pharmaceutical composition which, when administered as part of a desired dosage regimen (to a mammal, preferably a human) alleviates a symptom, ameliorates a condition, or slows the onset of disease conditions according to clinically acceptable standards for the disorder or condition to be treated, e.g., at a reasonable benefit/risk ratio applicable to any medical treatment.

### Administration

Compounds of the invention and their pharmaceutical compositions prepared as described herein can be administered according to the methods described herein through use of various forms, depending on the disorder to be treated and the age, condition, and body weight of the patient, as is well known in the art. As is consistent, recommended and required by medical authorities and the governmental registration authority for pharmaceuticals, administration is ultimately provided under the guidance and prescription of an attending physician whose wisdom, experience and knowledge control patient treatment.

For example, where the Compounds are to be administered orally, they may be formulated as tablets, capsules, granules, powders, or syrups; or for parenteral administration, they may be formulated as injections (intravenous, intramuscular, subcutaneous or intrathecal), drop infusion preparations, or suppositories. For application by the ophthalmic mucous membrane route or other similar transmucosal route, they may be formulated as drops or ointments.

These formulations for administration orally or by a transmucosal route can be prepared by conventional means, and if desired, the active ingredient may be mixed with any conventional additive or excipient, such as a binder, a disintegrating agent, a lubricant, a corrigent, a solubilizing agent, a suspension aid, an emulsifying agent, a coating agent, a cyclodextrin, and/or a buffer. Although the dosage will vary depending on the symptoms, age and body weight of the patient, the gender of the patient, the nature and severity of the disorder to be treated or prevented, the route of administration and the form of the drug, in general, a daily dosage of from 0.0001 to 2000 mg, preferably 0.001 to 1000 mg, more preferably 0.001 to 500 mg, especially more preferably 0.001 to 250 mg, most preferably 0.001 to 150 mg of the Compound is recommended for an adult human patient, and this may be administered in a single dose or in divided doses. Alternatively, a daily dose can be given according to body weight such as 1 nanogram/kg (ng/kg) to 200 mg/kg, preferably 10 ng/kg to 100 mg/kg, more preferably 10 ng/kg to 10 mg/kg, most preferably 10 ng/kg to 1 mg/kg. The amount of active ingredient which can be combined with a carrier material to produce a single dosage form will generally be that amount of the compound which produces a therapeutic effect.

The precise time of administration and/or amount of the Compounds and/or pharmaceutical compositions that will yield the most effective results in terms of efficacy of treatment in a given patient will depend upon the activity, pharmacokinetics, and bioavailability of a particular compound, physiological condition of the patient (including age, sex, disease type and stage, general physical condition, responsiveness to a given dosage, and type of medication), route of administration, etc. However, the above guidelines can be used as the basis for fine-tuning the treatment, e.g., determining the optimum time and/or amount of administration, which will require no more than routine experimentation consisting of monitoring the subject and adjusting the dosage and/or timing.

The phrase “pharmaceutically acceptable” is employed herein to refer to those excipients, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

### Pharmaceutical Compositions Incorporating ALS Compounds of Formula I

The pharmaceutical compositions of the invention incorporate embodiments of ALS Compounds of Formula I useful for methods of the invention and a pharmaceutically acceptable carrier. The compositions and their pharmaceutical compositions can be administered orally, topically, parenterally, by inhalation or spray or rectally in dosage unit formulations. The term parenteral is described in detail below. The nature of the pharmaceutical carrier and the dose of these ALS Compounds depend upon the route of administration chosen, the effective dose for such a route and the wisdom and experience of the attending physician.

A “pharmaceutically acceptable carrier” is a pharmaceutically acceptable material, composition, or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose, and sucrose; (2) starches, such as corn starch, potato starch, and substituted or unsubstituted (3-cyclodextrin; (3) cellulose, and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen free water; (17) isotonic saline; (18) Ringer's solution; (19) ethyl alcohol; (20) phosphate buffer solutions; and (21) other non-toxic compatible substances employed in pharmaceutical formulations.

Wetting agents, emulsifiers, and lubricants, such as sodium lauryl sulfate and magnesium stearate, as well as coloring agents, release agents, coating agents, sweetening, flavoring, and perfuming agents, preservatives and antioxidants can also be present in the compositions. Examples of pharmaceutically acceptable antioxidants include: (1) water soluble antioxidants, such as ascorbic acid, cysteine hydrochloride, sodium bisulfate, sodium metabisulfite, sodium sulfite, and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert matrix, such as gelatin and glycerin, or sucrose and acacia) and/or as mouthwashes, and the like, each containing a predetermined amount of a compound of the invention as an active ingredient. A composition may also be administered as a bolus, electuary, or paste.

In solid dosage form for oral administration (capsules, tablets, pills, dragees, powders, granules, and the like), a compound of the invention is mixed with one or more pharmaceutically acceptable carriers, such as sodium citrate or dicalcium phosphate, and/or any of the following:
(1) fillers or extenders, such as starches, cyclodextrins, lactose, sucrose, glucose, mannitol, and/or silicic acid;
(2) binders, such as, for example, carboxymethylcellulose, alginates, gelatin, polyvinyl pyrrolidone, sucrose, and/or acacia;
   (3) humectants, such as glycerol;
   (4) disintegrating agents, such as agar-agar, calcium carbonate, potato or tapioca starch, alginic acid, certain silicates, and sodium carbonate;
   (5) solution retarding agents, such as paraffin;
   (6) absorption accelerators, such as quaternary ammonium compounds;
   (7) wetting agents, such as, for example, acetyl alcohol and glycerol monostearate; (8) absorbents, such as kaolin and bentonite clay;
   (9) lubricants, such a talc, calcium stearate, magnesium stearate, solid polyethylene glycols, sodium lauryl sulfate, and mixtures thereof; and
   (10) coloring agents. In the case of capsules, tablets, and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols, and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered inhibitor(s) moistened with an inert liquid diluent.

Tablets, and other solid dosage forms, such as dragees, capsules, pills, and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes, and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner.

Examples of embedding compositions which can be used include polymeric substances and waxes. A compound of the invention can also be in micro-encapsulated form, if appropriate, with one or more of the above-described excipients.

Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as, for example, water or other solvents, solubilizing agents, and emulsifiers such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols, and fatty acid esters of sorbitan, and mixtures thereof.

Besides inert diluents, the oral compositions can also include adjuvants such as wetting agents, emulsifying and suspending agents, sweetening, flavoring, coloring, perfuming, and preservative agents.

Suspensions, in addition to the active inhibitor(s) may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing one or more inhibitor(s) with one or more suitable nonirritating excipients or carriers comprising, for example, cocoa butter, polyethylene glycol, a suppository wax or a salicylate, which is solid at room temperature, but liquid at body temperature and, therefore, will melt in the rectum or vaginal cavity and release the active agent.

Formulations which are suitable for vaginal administration also include pessaries, tampons, creams, gels, pastes, foams, or spray formulations containing such carriers as are known in the art to be appropriate.

Dosage forms for the topical or transdermal administration of an inhibitor(s) include powders, sprays, ointments, pastes, creams, lotions, gels, solutions, patches, and inhalants. The active component may be mixed under sterile conditions with a pharmaceutically acceptable carrier, and with any preservatives, buffers, or propellants which may be required.

The ointments, pastes, creams, and gels may contain, in addition to a compound of the invention, excipients, such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to a compound of the invention, excipients such as lactose, talc, silicic acid, aluminum hydroxide, calcium silicates, and polyamide powder, or mixtures of these substances. Sprays can additionally contain customary propellants, such as chlorofluorohydrocarbons and volatile unsubstituted hydrocarbons, such as butane and propane.

A compound useful for application of methods of the invention can be alternatively administered by aerosol. This is accomplished by preparing an aqueous aerosol, liposomal preparation, or solid particles containing the composition. A nonaqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of a compound of the invention together with conventional pharmaceutically acceptable carriers and stabilizers. The carriers and stabilizers vary with the requirements of the particular composition, but typically include nonionic surfactants (Tweens, Pluronics, sorbitan esters, lecithin, Cremophors), pharmaceutically acceptable co-solvents such as polyethylene glycol, innocuous proteins like serum albumin, oleic acid, amino acids such as glycine, buffers, salts, sugars, or sugar alcohols. Aerosols generally are prepared from isotonic solutions.

Transdermal patches have the added advantage of providing controlled delivery of a compound of the invention to the body. Such dosage forms can be made by dissolving or dispersing the agent in the proper medium. Absorption enhancers can also be used to increase the flux of the inhibitor(s) across the skin. The rate of such flux can be controlled by either providing a rate controlling membrane or dispersing the inhibitor(s) in a polymer matrix or gel.

Pharmaceutical compositions of this invention suitable for parenteral administration comprise one or more compounds of the invention in combination with one or more pharmaceutically acceptable sterile aqueous or nonaqueous solutions, dispersions, suspensions or emulsions, or sterile powders which may be reconstituted into sterile injectable solutions or dispersions just prior to isotonic with the blood of the intended recipient or suspending or thickening agents. Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include tonicity-adjusting agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a compound useful for practice of methods of the invention, it is desirable to slow the absorption of the compound from subcutaneous or intramuscular injection. For example, delayed absorption of a parenterally administered drug form is accomplished by dissolving or suspending the drug in an oil vehicle.

Injectable depot forms are made by forming microencapsule matrices of inhibitor(s) in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

The pharmaceutical compositions may be given orally, parenterally, topically, or rectally. They are, of course, given by forms suitable for each administration route. For example, they are administered in tablets or capsule form, by injection, inhalation, eye lotion, ointment, suppository, infusion; topically by lotion or ointment; and rectally by suppositories. Oral administration is preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection, and infusion.

The pharmaceutical compositions of the invention may be “systemically administered” “administered systemically,” “peripherally administered” and “administered peripherally” meaning the administration of a ligand, drug, or other material other than directly into the central nervous system, such that it enters the patient's system and thus, is subject to metabolism and other like processes, for example, subcutaneous administration.

The compound(s) useful for application of the methods of the invention may be administered to humans and other animals for therapy by any suitable route of administration, including orally, nasally, as by, for example, a spray, rectally, intravaginally, parenterally, intracisternally, and topically, as by powders, ointments or drops, including buccally and sublingually.

Regardless of the route of administration selected, the compound(s) useful for application of methods of the invention, which may be used in a suitable hydrated form, and/or the pharmaceutical compositions of the present invention, are formulated into pharmaceutically acceptable dosage forms by conventional methods known to those of skill in the art.

Actual dosage levels of the compound(s) useful for application of methods of the invention in the pharmaceutical compositions of this invention may be varied so as to obtain an amount of the active ingredient which is effective to achieve the desired therapeutic response for a particular patient, composition, and mode of administration, without being toxic to the patient.

The concentration of a compound useful for application of methods of the invention in a pharmaceutically acceptable mixture will vary depending on several factors, including the dosage of the compound to be administered, the pharmacokinetic characteristics of the compound(s) employed, and the route of administration.

In general, the compositions useful for application of methods of this invention may be provided in an aqueous solution containing about 0.1-10% w/v of a compound disclosed herein, among other substances, for parenteral administration. Typical dose ranges are those given above and may preferably be from about 0.001 to about 500 mg/kg of body weight per day, given in 1-4 divided doses. Each divided dose may contain the same or different compounds of the invention. The dosage will be an effective amount depending on several factors including the overall health of a patient, and the formulation and route of administration of the selected compound(s).

### EXPERIMENTAL EXAMPLES

### Materials and Methods

### Quantification & Statistical Analysis

All quantification and statistical analyses (completed in GraphPad Prism version 8) were completed as described in the figure legends and in the methods. In brief, the statistical analysis for all experiments completed in c9ALS/FTD patient-derived cells accounted for repeated measurements of the same patient cell line using a Repeated Measures One- or Two-way ANOVA. Tukey's multiple comparison test was used to compare multiple samples as indicated in figure legends. For studies completed in vitro or in HEK293T cells, statistical significance was determined by using a One-way ANOVA or t-test as indicated. For all panels where statistical significance is indicated, * P<0.05, ** P<0.01, *** P<0.001, and **** P<0.0001. Bar graphs display individual data points and reported as the mean±SD. All compound-treated samples were normalized to vehicle unless otherwise noted. The DMSO concentration for vehicle-treated samples was always 0.1%.

### Biochemical & Biophysical Methods

### General.

RNAs and 5′-biotinylated oligonucleotides were purchased from Dharmacon Inc. (GE Healthcare). All oligos were deprotected as outlined in the manufacturer's protocols and were subsequently desalted via PD-10 columns (GE Healthcare). Concentrations of oligonucleotides were determined by UV/VIS spectrometry using a Beckman Coulter DU 800 spectrophotometer. Absorbance was measured at 260 nm at 90° C. (extinction coefficients for RNAs were provided by the vendor). Structures and sequences of RNA hairpins reported in this study can be found in Table 1. DNA oligonucleotides were obtained from Integrated DNA Technologies (IDT) and standard desalting was provided by the manufacturer. These oligos were used without further purification. Sequences of DNA oligonucleotides (including primers) used in this study can be found in Table 2.

### In Vitro Methods

### Affinity Measurements by Microscale Thermophoresis (MST).

Affinity measurements were performed by MST using a Monolith NT.115 system (NanoTemper Technologies) with 5′-Cy5-labeled r(G₄C₂)₈ (SEQ ID NO: 1), 5′-Cy5-labeled d(G₄C₂)₈ (SEQ ID NO: 1), and 5′-Cy5-labeled base pair control r(G₂C₂)₄GAAA(G₂C₂)₄ (SEQ ID NO: 49). All oligos were deprotected according to the manufacturer's recommended protocol. Samples were prepared as previously described³⁰. Briefly, RNA or DNA (5 nM) was folded in 1×MST Buffer (8 mM Na₂HPO₄, 185 mM NaCl, 1 mM EDTA) by heating at 95° C. for 5 minutes and slowly cooling to room temperature. Tween-20 was then added to a final concentration of 0.05% (v/v). Serial dilutions of compound in 1×MST Buffer containing 5 nM folded nucleic acid were then carried out to yield the desired compound concentrations. Samples were incubated for 90 minutes at room temperature and then loaded into premium capillaries (NanoTemper Technologies). The following parameters were used on the Monolith NT.115 system: 10% LED, 20-80% MST power, Laser-On time=30 s, Laser-Off time=5 seconds. Fluorescence was detected using excitation wavelengths of 605-645 nm and emission wavelengths of 680-685 nm. The resulting data were analyzed by thermophoresis analysis and fit using the quadratic binding equation in the MST analysis software (NanoTemper Technologies). The dissociation constant was determined using Equation 1. The reported K_{d} values are an average of three independent experiments.

### Eq. 1

$${K}_{d} = \frac{unbound + ( bound - unbound )}{2} * ( [ RNA ] + [ 2 b ] + Kd \sqrt{{( [ RNA ] + [ 2 b ] + K d )}^{2} - 4 ( [ RNA ] * [ 2 b ] )}$$

### In Vitro RIBOTAC Cleavage Mapping: Gel Analysis.

The 5′ end of r(G₄C₂)₈ (SEQ ID NO: 1) (a model of r(G₄C₂)^{exp}) was radiolabeled with ³²P and purified on a denaturing polyacrylamide gel (15%), as previously described^{31,32}. After purification, labeled RNA (25 nM) was folded in 1×RNase L buffer (25 mM Tris-HCl, pH 7.4, and 100 mM NaCl) for 5 minutes at 95° C. The folded RNA was cooled to room temperature and 2-mercaptoethanol (7 mM final concentration), ATP (50 μM final concentration), and MgCl₂ (10 mM final concentration) were added to the solution. Dilutions of compound in the folded RNA solution were then made and incubated for 15 minutes at room temperature. After the incubation, RNase L was added to a final concentration 50 nM. Samples were incubated at 37° C. overnight. Cleavage fragments were then separated on a denaturing 15% polyacrylamide gel, imaged by autoradiography (Typhoon FLA 9500), and quantified using the QuantityOne software (BioRad). All experiments were performed in duplicate.

### Cellular Methods

### Cell Culture.

HEK293T cells (CRL-3216) were acquired from American Type Culture Collection (ATCC): CRL-3216 (female, fetus). HEK293T cells were maintained at 37° C. and 5% CO₂ in Dulbecco's Modified Eagle Medium (DMEM; Corning) supplemented with 10% fetal bovine serum (FBS; Sigma Aldrich), 1% penicillin-streptomycin (PS; Corning) and 1% glutagro supplement (Corning).

Patient-derived lymphoblastoid cell lines (LCLs) were acquired from the Coriell Institute: ND11583 (male, age 59, with GGGGCC expansion); ND12438 (male, age 65, with GGGGCC expansion); ND09492 (male, age 52, with GGGGCC expansion); and GM07491 (male, age 17, healthy). LCLs were maintained at 37° C. and 5% CO₂ in Roswell Park Memorial Institute (RPMI) 1640 Medium supplemented with 10% (v/v) FBS, 1% (v/v) Penicillin-Streptomycin solution (Life Technologies), and 1% (v/v) Glutagro (Life Technologies).

ALS and healthy patient-derived induced pluripotent stem cell (iPSC) lines were acquired from the Laboratory for Neurodegenerative Research, Johns Hopkins University School of Medicine: CSOBUU (female, age 63, with GGGGCC expansion); CS7VCZ (male, age 64, with GGGGCC expansion); CSONKC (female, age 60, with GGGGCC expansion); CS2YNL (male, age 60, with GGGGCC expansion); CS8PAA (female, age 58, healthy); CS9XH7 (male, age 53, healthy); EDi044-A (female, age 80, healthy); and CS1ATZ (male, age 60, healthy). iPSCs were maintained in mTeSR™ 1 feeder-free medium (Basal medium) (STEMCELL Technologies; Catalog #85850), in Matrigel-(Corning, Catalog #356234) coated plates, according to STEMCELL's protocols. Compound treatment of iPSCs was carried out for 4 days in Basal medium in Matrigel-coated 6-well plates. On Days 1 and 3, the medium was removed and fresh medium containing compound was added to each well. The final concentration of DMSO in all samples was 0.1% (v/v).

Differentiated motor neurons (iPSNs) were derived from the iPSCs listed in the previous section. The differentiation process was based on a previously reported method with slight modifications^{33,34}. Briefly, iPSCs were plated into Matrigel-coated 100 mm dishes (30-40% confluence). Neuroepithelial induction was performed by replacing the Basal medium with Stage 1 medium [47.5% IMDM (Iscove's Modified Dulbecco's Medium), 47.5% F12 medium, 1% NEAA (Non-Essential Amino Acids) (Life Technologies), 2% B27 (Invitrogen), 1% N2 (Invitrogen), 1% PSA (Penicillin-Streptomycin-Amphotericin), 0.2 μM LDN193189 (Stemgent), 10 μM SB431542 (STEMCELL Technologies) and 3 μM CHIR99021 (Sigma-Aldrich)]. The medium was changed daily for 6 days, after which cells were detached from plates using Accutase (STEMCELL Technologies). Cells were seeded into 6-well plates (1.5×10⁶ cells/well) in 3 mL of Stage 2 medium [Stage 1 medium supplemented with 0.1 μM All-trans RA (Sigma-Aldrich) and 1 μM SAG (Cayman Chemicals)]. Cells were maintained in Stage 2 medium, with daily medium changes, through Day 11 of the differentiation process. On Day 12, the Stage 2 cell medium was removed and replaced with Stage 3 medium [47.5% IMDM, 47.5% F12 medium, 1% NEAA, 2% B27, 1% N2, 1% PSA, 0.1 μM Compound E (Millipore; Catalog #: 565790), 2.5 μM DAPT (Sigma-Aldrich), 0.1 μM db-cAMP (Millipore), 0.5 μM All-trans RA, 0.1 μM SAG, 20 ng/mL ascorbic acid, 10 ng/mL BDNF (STEMCELL Technologies), and 10 ng/mL GDNF (STEMCELL Technologies)]. This medium was replaced every three days until the end of the differentiation process. Typically, a minimum of 18 days is required to produce immature differentiated motor neurons; 30 days of differentiation is the standard for producing mature differentiated spinal neurons. The cells can be maintained in medium for an additional 14-28 days after reaching maturation.

Differentiated motor neurons were treated with compound starting at Day 15. Cells were treated in Matrigel coated 6-well plates with Stage 3 differentiation medium (see above) supplemented with compound. Cells were treated with fresh medium containing compound diluted in 0.1% DMSO every 3-4 days until reaching full maturity at Day 32. On Day 32 the cells were harvested for analysis.

### Cell Viability.

Patient-derived LCLs were seeded overnight in 96-well plates (˜10⁴ cells/well) and treated with compounds for 96 hours. Cell viability was measured using the CellTiter-Fluor™ Cell Viability Assay (Promega) per the manufacturer's protocol. iPSC cell viability was measured by seeding cells in Matrigel-coated 6-well plates with Basal medium (iPSC maintenance described above). After overnight incubation, the medium was replaced with fresh medium containing compounds diluted in 0.1% DMSO. Cells were treated with compound for 96 hours. Cell viability was measured using the AlamarBlue™ Cell Viability Reagent (DAL1025, Thermo Fisher Scientific) per the manufacturer's protocol. iPSN cell viability was measured by

### Measuring RAN Translation in HEK Cells.

HEK293T cells were cultured according to the methods described above. After reaching ˜80% confluency, HEK293T cells were batch-transfected, according to the manufacturer's protocol, in 100 mm dishes using the Lipofectamine 3000 transfection system (Thermo Fischer) for 5 hours with 2.5 g of a plasmid encoding r(G₄C₂)₆₆-no ATG-nano-luciferase and 1 μg of a plasmid encoding SV40-Firefly luciferase (Life Technologies). Cells were seeded into a 384-well plate and incubated overnight. Compounds were treated in <0.1% DMSO final concentration for 24 hours. RAN and canonical translation were measured using a Dual-Luciferase® Reporter Assay System (Promega) following the manufacturer's protocol. Experiments were performed as biological triplicates (n=3). Fluorescence intensities in untransfected cells were measured to determine the background signal.

Measuring Levels of C9orf72 and C9orf72 Variants by RT-qPCR. LCLs and iPSCs were seeded in 6-well plates (˜10⁶ cells in 2 mL of Basal medium) and incubated overnight at 37° C. with 5% CO₂. LCLs were then treated with compound for 4 days without media changes; patient-derived iPSCs were seeded at ˜80% confluency and treated, as described above. Treatment with either a G₄C₂-ASO or Control-ASO (100 nM) was used as a control. ASO transfection was achieved using Lipofectamine RNAiMax (Life Technologies), following the manufacturer's protocol. After 4 days of treatment (with either compound or ASO), total RNA was extracted using the Quick-RNA Miniprep Kit (Zymo Research). RNA was quantified via Nanodrop and ˜1 μg of total RNA was reverse transcribed using qScript (Quantbio), according to the manufacturer's guidelines. RT-qPCR was performed on a QuantStudio™ Real-Time PCR Instrument (Applied Biosystems) using Power SYBR Green Master Mix (Applied Biosystems). Expression levels of mRNAs were normalized to GAPDH. See Table 2 for a list of primers used.

For iPSNs cells were differentiated, following the protocol outlined above, until Day 15. Beginning on Day 15, media containing compound in 0.1% DMSO was replenished on cells every 3-4 days until Day 32 of the differentiation process. RNA was extracted as described above and the expression levels of C9orf72 variants was determined by RT-qPCR and normalized to GAPDH. See Table 2 for a list of primers used.

siRNA Experiments.

iPSCs were cultured as described in the “Cell Culture,” section. Briefly, iPSCs were plated into 6-well Matrigel coated plates and treated with compound and siRNA for four days. On day one, fresh media was added to the cells and siRNAs were transfected using Lipofectamine RNAiMax (Life Technologies), following the manufacturer's protocol. Cells were incubated for 1 hour at 37° C. and then compound was added to a final concentration of 50 nM (DMSO 0.1% (v/v)). Cells were incubated for 48 h, then media was replaced and the siRNA re-transfected. Following a 1 hour incubation, cells were treated with compound and then incubated for another 48 hours. After a total of 96 hours of treatment, RNA was extracted and RT-qPCR was performed following the protocol outline in the “Measuring Levels of C9orf72 and C9orf72 Variants by RT-qPCR,” section.

### Transcriptome-Wide Studies Via RNA-Seq.

Total RNA integrity was confirmed by Agilent 2100 Bioanalyzer RNA nano chip, and the quantity was measured by Qubit 2.0 Fluorometer (Invitrogen). The library preparation was performed using NEBNext Ultra II Directional RNA kit (NEB, E7760) in combination with NEBNext rRNA depletion module (NEB, E6310) and RNA fragmentation module (NEB, E6150S), following manufacturer's recommendations. Briefly, a total of 200 ng RNA was first processed with depleted of ribosomal RNA, and then randomly fragmented to achieve range between 150 to 300 nucleotides. The fragmented RNAs were random primed for the first-strand synthesis, and the second strand was synthesized with dUTPs. The strand information is thus preserved by using USER enzyme (Uracil-specific excision reagent). The cDNA was PCR amplified and pooled equimolar to load onto the NextSeq 500 v2.5 flow cell and sequenced with 2×40 bp paired-end method. The output fastq files were aligned using STAR 3⁵. The read counts of specific regions were extracted using samtools.³⁶ The global differential gene expression analysis was performed using featureCounts and Deseq2.^{37,38}

Measuring poly(GP) Abundance by Electrochemiluminescence Assay.

Meso Scale Discovery (MSD) Technology utilizes electrochemiluminescence to detect biomarkers, such as poly(GP), in protein samples. All cells were seeded in 6-well plates. LCLs were seeded in 2 mL of RPMI at 1×10⁶ cells/mL and treated with compound for 4 days without media changes; patient-derived iPSCs were seeded at ˜80% confluency and treated as described above; patient-derived iPSNs began compound treatment on Day 15 of the differentiation process as previously described. Compounds were diluted to 0.1% DMSO final concentration in medium. After 4 days of compound treatment, cells were pelleted and protein was extracted using CoIP buffer (50 mM Tris-HCL, pH 7.4, 300 mM NaCl, 5 mM EDTA, 1% Triton-X 100, 2% sodium dodecyl sulfate, 0.01% protease and phosphatase inhibitors) for 5 minutes on ice. Protein was then sheared by sonication (3 second intervals at 35% power for ˜20 seconds of total “ON” interval time). Detergent from the CoIP buffer was removed using a Pierce™ Detergent Removal Spin Column 0.5 mL (Thermo Scientific) according to the manufacturer's protocol. Protein samples were quantified using a Pierce™ Micro BCA Protein Assay Kit (Thermo Scientific).

Poly(GP) levels were measured by an electrochemical luminescent sandwich immunoassay. MSD Gold 96-well small spot streptavidin SECTOR plates (MSD Technology) were incubated with 2 μg/ml of Biotin antibody overnight at 4° C.

Following incubation wells were washed three times in 1×TBST (Tris-buffered saline containing 0.1% (v/v) Tween-20) and blocked with a 3% (w/v) BSA solution in 1×TBST for 1 hour with shaking at room temperature. The wells were washed three times with 1×TBST and 80 μg of cell lysate were added to each well. Wells were incubated with lysate for 2 hours with shaking at room temperature. Following protein incubation, the wells were washed three times with 1×TBST and 4 μg/mL of Sulfo labeled antibody, diluted in 3% BSA solution in 1×TBST, were added to the wells. The wells were incubated at room temperature, covered in aluminum foil with shaking for 1 hour. After incubation, the wells were washed three times with 1×TBST and 150 μL of 1×MSD GOLD Read Buffer (MSD Technology) was applied to the wells immediately before reading the plate. The plate was read using a SECTOR Imager (MSD Technology).

### Western Blotting.

Protein samples were prepared as described for analysis by the electrochemical luminescent sandwich immunoassay. Approximately 20 μg total protein was loaded onto a 4-20% Mini-PROTEAN® TGX™ Precast Protein Gel (Bio-Rad) and run at 130 V for 1 hour in Tris-Glycine/SDS running buffer (25 mM Tris base, 190 mM glycine, 0.1% SDS, pH 8.3). Following electrophoresis, the protein was transferred to a PVDF membrane using Tris-Glycine transfer buffer (25 mM Tris base, 190 mM glycine, 20% methanol, pH 8.3). After transfer, the membrane was washed with 1×TBST and blocked in a solution of 5% (w/v) milk in 1×TBST for 30 minutes at room temperature with shaking. The membrane was then incubated in 1:3000 C9ORF72 primary antibody (GeneTex, GTX119776) in 1×TBST containing 5% milk overnight at 4° C. The membrane was washed three times with 1×TBST and incubated with 1:2000 anti-mouse IgG horseradish-peroxidase secondary antibody conjugate (Cell Signaling Technology) in 1×TBST for 1 hour at room temperature with gentle shaking. The membrane was then washed three times with 1×TBST and protein expression was quantified using SuperSignal West Pico Plus Chemiluminescent Substrate (Life Technologies), per the manufacturer's protocol, and film exposure. To quantify β-actin expression, the membrane was washed with 1×TBST and stripped using 1×Stripping Buffer (200 mM glycine, 1% (v/v) Tween-20, 0.1% (w/v) SDS, pH 2.2). Following stripping, the membrane was washed with 1×TBST and again blocked in a 5% milk solution in 1×TBST at room temperature with shaking for 30 minutes. The membrane was then incubated with 1:3000 β-actin primary antibody (Cell Signaling Technology) in 1×TBST containing 5% milk overnight at 4° C. The membrane was washed with 1×TBST and incubated with 1:5,000 anti-mouse IgG horseradish-peroxidase secondary antibody conjugate (Cell Signaling Technology) in 1×TBST at room temperature for 1 hour. β-actin protein expression was quantified as previously described.

### In Vivo Methods

Therapeutic Efficacy in c9ALS/FTD Mouse Models.

All animal studies were approved by the Scripps Florida Institutional Animal Care and Use Committee.

### PWR500 BAC Mice.

A total of 20 age- and gender-matched mice (12 +/+PWR500 [5 Male and 7 Female] and 8 WT [4 Male and 4 Female]), ranging from 18-21 weeks old were used in therapeutic efficacy studies (Table 4). Mice were treated with a single bolus injection of 33 nmol of compound 2 formulated in 1% (v/v) DMSO/99% water, administered by an intracerebroventricular injection (ICV). Three weeks post injection, mice were euthanized, and tissue was harvested for study.

### Measuring C9orf72 Variants by RT-qPCR.

Postmortem brain tissue was harvested and sliced along the sagittal plane at the midline. The left hemisphere was frozen for RNA and protein analysis. Frozen brain tissue was homogenized in 300 μL Tris-EDTA buffer with a 1:5 w/v ratio of 2× Protease and Phosphatase Inhibitors. RNA was extracted from half of the homogenized tissue solution (150 μL) using Triazol LS (added at a 1:3 ratio to the homogenized tissue). The Triazol/tissue solution was centrifuged for 15 minutes at 16,000 rpm and the supernatant was collected. An equal volume of 100% ethanol was added to the supernatant, and RNA was purified using the Direct-zol RNA Kit (Zymo Research) per the manufacturer's protocol. RT-qPCR was then performed as described above. Expression levels of mRNAs were normalized to mouse β-actin. See Table 2 for a list of primers.

Measuring poly(GP) Abundance by Electrochemiluminescence Assay.

Postmortem brain tissue was harvested and sliced along the sagittal plane at the midline. The left hemisphere was frozen for RNA and protein analysis. Frozen brain tissue was homogenized in 300 μL Tris-EDTA buffer with a 1:5 w/v ratio of 2× protease and phosphatase inhibitors, as was done for RNA analysis of the tissue. Half of the homogenized tissue (150 μL) was mixed with 2× Lysis Buffer (50 mM Tris, pH 7.4, 250 mM NaCl, 2% (v/v) Triton X-100, and 4% (w/v) SDS) and sonicated at 1 second on/off intervals at 30% for a total of 15 seconds. Detergent from was removed using a Pierce™ Detergent Removal Spin Column 0.5 mL (Thermo Scientific) according to the manufacturer's protocol. Protein concentrations were then measured by BCA assay (Pierce Biotechnology) and poly(GP) was measured as described above for the electrochemical luminescent sandwich immunoassay.

### Immunohistochemistry (IHC).

Postmortem brain tissue was harvested and sliced along the sagittal plane at the midline. The right hemisphere was stored for 48 hours in 10% neutral buffered formalin. Tissue processing, embedding and sectioning were then performed by the Scripps Florida Histology Core. Formalin-fixed tissue was processed on a Sakura Tissue-Tek VIP 5 paraffin processor. Tissue was first embedded in paraffin, sectioned at 4 μm, and then mounted on positively charged slides. Slides were stained with primary antibody (see below) using the Leica BOND-MAX platform. Slides were then subjected to the Leica Refine Detection Kit containing the secondary polymer, DAB chromagen, and the counterstain. Slides were dehydrated in graded alcohols and cleared in xylene, before being cover slipped with a permanent mounting medium (Cytoseal 60; Thermo Scientific).

### Antibodies.

NeuN (1:2000, RRID: AB_177621 Millipore); poly-GA (1:2000, MABN889, Millipore); poly-GP (1:5000, ABN455, Millipore); Calbindin (1:5000, RRID: AB_476894, Millipore); TDP-43 (1:2000, RRID: AB_615042, Proteintech).

RNA Fluorescence In Situ Hybridization (FISH) with Immunofluorescence (IF).

Postmortem brain tissue was excised and sliced along the sagittal plane at the midline. The right hemisphere was flash frozen with Optimal Cutting Temperature (OCT) compound in 2-methylbutane in liquid nitrogen. Frozen tissue was sectioned (10 m) using a cryostat and slides were stained as previously described.³⁹ Briefly, frozen sections were fixed in 4% paraformaldehyde in 1×DPBS for 20 minutes then incubated in ice cold 70% ethanol at 4° C. for 30 minutes. Once fixed, slides were incubated for 10 minutes in 40% formamide in 2×SSC Buffer at room temperature. Slides were blocked in 1× Hybridization Buffer (40% formamide, 2×SSC Buffer, 20 g/mL BSA, 100 mg/mL dextran sulfate, 250 μg/mL tRNA, and 2 mM vanadyl sulfate) for 30 minutes at 55° C. Slides were then incubated for 3 hours at 55° C. with 200 ng/mL of FISH probe (Table 1) in 1× Hybridization Buffer. After hybridization, slides were washed in 40% formamide in 2×SSC Buffer (three times) and 1×DPBS. Slides were then co-stained with NeuN (Sigma: MAB377B) as follows. Tissue was permeabilized for 15 minutes with 0.5% (v/v) Triton X-100 in 1×DPBS at 4° C. The tissue was then blocked with 2% (v/v) goat serum in 1×DPBS for 1.5 hours at 4° C. Slides were incubated overnight at 4° C. with NeuN (1:500, MAB377B, Sigma) diluted in 2% goat serum in 1×DPBS. After incubation with the primary antibody, slides were washed three times with 1×DPBS and incubated with the secondary antibody (donkey anti-goat IgG conjugated to Alexa Fluor 488; AbCam Inc) (diluted 1:500 in 1×DPBS) for 1 hour at room temperature. Slides were then washed three times with 1×DPBS and quenched with 0.25% Sudan Black B (Millipore) diluted in 70% ethanol. After slides dried completely, they were mounted with mounting medium containing DAPI (Invitrogen) and imaged using a 60× objective.

### Computational Methods

### Parameterization ALS Compound Formula I.

All Amber force field parameters of ALS Compound Formula I were obtained from previous study (table below)³¹. The force field parameters of the NH-modified PEG linker (NH-PEG; below) were prepared as previously described⁴⁰⁻⁴⁴. The AMBER GAFF force field,⁴³ was used to define the atom types while RESP charges were derived following the multimolecular RESP charge fitting protocol.^{44,45} The molecules were optimized and the electrostatic potentials as a set of grid points were calculated at the HF level using the 6-31G* basis set, where Gaussian09,⁴⁶ was used to perform these calculations.

### Binding Study.

The binding modes of ALS Compound Formula I to a model of r(G₄C₂) repeats were determined by previously established methods.³¹ The lowest energy binding modes were used to homology model the ALS Compound Formula 1 bound to a duplex model of r(G₄C₂) repeats.

### Explicit Solvent Molecular Dynamics (MD) Simulation.

Explicit water MD simulations were performed to find optimal bound conformations. The initial coordinates for MD simulations were extracted from the results of homology modeling, and 21 Na⁺ ions,⁴⁷ were added to make the system neutral. TIP3P water molecules were added to the systems so that all the atoms of RNA and 3 were at least 8.0 Å away from the edge of the simulation box. Long-range electrostatic interactions were calculated using the Particle Mesh Ewald method.⁴⁸ Temperature and the pressure were maintained through the simulations as 300 K and 1 bar using Langevin dynamics and Berendsen barostat. Three independent MD simulations for 500 ns with a time step of 1 fs were completed. The total of 1.5 s combined MD trajectories were produced and used in cluster analysis.

### Cluster Analysis and MM-PBSA Calculation.

Cluster analysis was conducted to determine structure population using CPPTRAJ. CPPTRAJ groups similar conformations together in the a given trajectory file by Root-mean-square deviation (RMSD) analysis. The density-based scanning algorithm was used with RMSD cutoff distance of 1.3 Å to form a cluster. Cluster analysis revealed three stable binding conformations. MM-PBSA analyses were conducted on each cluster to determine the lowest binding free energy states. The MMPBSA.py module of AMBER16 was used and the results of relative binding free energies for are presented. The binding conformations with the lowest binding energies were selected as the most stable binding conformations.

### Synthetic Methods

### Abbreviations:

Ac₂O, acetic anhydride; CDCl₃, chloroform-d; CD₃OD, methanol-d₄; Cs₂CO₃, Cesium carbonate; DIPEA, N,N-diisopropylethylamine; DCM, dichloromethane; DMF, N,N-dimethylformamide; DMSO, dimethyl sulfoxide; EDC, N-Ethyl-N′-(3-dimethylaminopropyl)carbodiimide hydrochloride; Et₃N, triethylamine; EtOAc, ethyl acetate; HCl, hydrochloric acid; H₂O, water; HOBt, 1-hydroxybenzotriazole; HPLC, high performance liquid chromatography; K₂CO₃, potassium carbonate; LiCl, lithium chloride; MALDI, matrix-assisted laser desorption/ionization; MeOH, methanol; NaH, sodium hydride; NaHCO₃, sodium bicarbonate; NaI, sodium iodide; NaOMe, sodium methoxide; Na₂SO₄, sodium sulfate; NMR, nuclear magnetic resonance; PEG, polyethylene glycol; TFA, trifluoroacetic acid; THF, tetrahydrofuran; TLC, thin layer chromatography.

### General.

Reagents and solvents were purchase from standard suppliers and used without further purification. Reactions were monitored by TLC. Spots were visualized with UV light or by phosphomolybdic acid or Ninhydrin staining. Products were purified by Isolera One flash chromatography system (Biotage) using pre-packed silica gel column (Agela Technologies) or by HPLC (Waters 2489 pump and 1525 detector) using a SunFire® Prep C18 OBD™ 5 μm column (19×150 mm) with the flow rate of 5 mL/minutes. Compound purity was analyzed by HPLC using a SunFire® C18 3.5 μm column (4.6 ×150 mm) with the flow rate of 1 mL/minutes. NMR spectra were measured by a 400 UltraShield™ (Bruker) (400 MHz for ¹H and 100 MHz for ¹³C) or Ascend™ 600 (Bruker) (600 MHz for ¹H and 150 MHz for ¹³C). Chemical shifts are expressed in ppm relative to trimethylsilane (TMS) for ¹H and residual solvent for ¹³C as internal standards. Coupling constant (J values) are reported in Hz. High resolution mass spectra were recorded on a 4800 Plus MALDI TOF/TOF Analyzer (Applied Biosystems) with α-cyano-4-hydroxycinnamic acid matrix and TOF/TOF Calibration Mixture (AB Sciex Pte. Ltd.) or an Agilent 1260 Infinity LC system coupled to an Agilent 6230 TOF (HR-ESI) equipped with a Poroshell 120 EC-C18 column (Agilent, 50 mm×4.6 mm, 2.7 μm).

### Synthetic Experimental Procedure

The synthesis of the carbazole compound, Formula II, the r(G₄C₂°)^{exp} binding compound, followed an established experimental route. Coupling of the carbazole Formula II to the RNase recruiting moiety, Formula IIIA was accomplished by attaching ethyl 4-bromo butanoate to the Carbazole Formula II with X as OH, hydrolyzing the ethyl ester group to form a carboxylic acid group and carbodiimide amide coupling the carboxylic acid group with the amine group of amine PEG RNase recruiting moiety Formula IIIA wherein the aminePEG group is coupled to the OR′ group of Formula IIIA by formation of the sodium phenoxide of the OR′ group of Formula IIIA and coupling phenoxide group with ω-bromo-α-tosyl PEG. The scheme for the synthesis of the ALS Compound Formula I with n as 2 and R as methyl is shown as following Scheme I

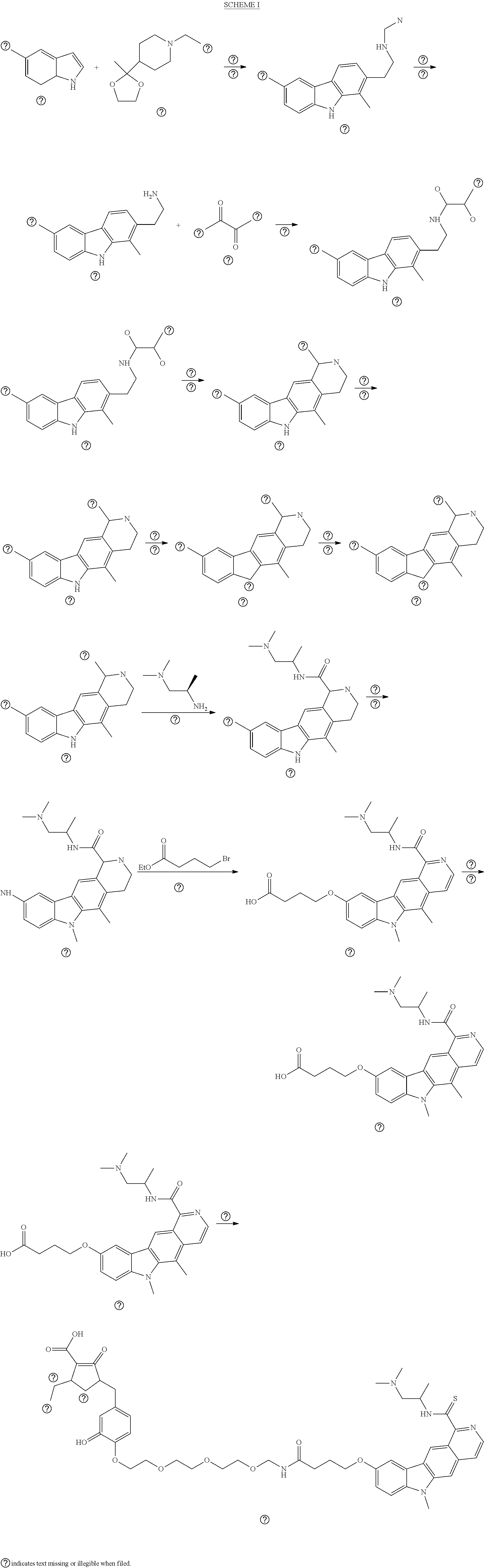

### REFERENCES

1 Bernat, V. & Disney, Matthew D. RNA Structures as Mediators of Neurological Diseases and as Drug Targets. Neuron 87, 28-46, doi:https://doi.org/10.1016/j.neuron.2015.06.012 (2015).
2 Todd, P. K. & Paulson, H. L. RNA-mediated neurodegeneration in repeat expansion disorders. Ann Neurol 67, 291-300, doi:10.1002/ana.21948 (2010).
3 Rohilla, K. J. & Gagnon, K. T. RNA biology of disease-associated microsatellite repeat expansions. Acta Neuropathologica Communications 5, 63, doi:10.1186/s40478-017-0468-y (2017).
4 DeJesus-Hernandez, M. et al. Expanded GGGGCC Hexanucleotide Repeat in Noncoding Region of C9ORF72 Causes Chromosome 9p-Linked FTD and ALS. Neuron 72, 245-256, doi:https://doi.org/10.1016/j.neuron.2011.09.011 (2011).
5 Renton, Alan E. et al. A Hexanucleotide Repeat Expansion in C9ORF72 Is the Cause of Chromosome 9p21-Linked ALS-FTD. Neuron 72, 257-268, doi:https://doi.org/10.1016/j.neuron.2011.09.010 (2011).
6 Liang, X.-H., Sun, H., Nichols, J. G. & Crooke, S. T. RNase H1-Dependent Antisense Oligonucleotides Are Robustly Active in Directing RNA Cleavage in Both the Cytoplasm and the Nucleus. Mol Ther 25, 2075-2092, doi:10.1016/j.ymthe.2017.06.002 (2017).
7 Swayze, E. E. et al. Antisense oligonucleotides containing locked nucleic acid improve potency but cause significant hepatotoxicity in animals. Nucleic Acids Res 35, 687-700, doi:10.1093/nar/gkl1071 (2007).
8 McMahon, B. M. et al. Pharmacokinetics and tissue distribution of a peptide nucleic acid after intravenous administration. Antisense Nucleic Acid Drug Dev 12, 65-70, doi:10.1089/108729002760070803 (2002).
9 Schoch, K. M. & Miller, T. M. Antisense Oligonucleotides: Translation from Mouse Models to Human Neurodegenerative Diseases. Neuron 94, 1056-1070, doi:10.1016/j.neuron.2017.04.010 (2017).
Swayze, E. et al. Kinetics of ASO Distribution and Pharmacodynamics in the CNS after an Intrathecal Bolus Dose in Rat (S38.008). Neurology 86, S38.008 (2016).
11 Wang, Z.-F. et al. The Hairpin Form of r(G4C2)exp in c9ALS/FTD Is Repeat-Associated Non-ATG Translated and a Target for Bioactive Small Molecules. Cell Chemical Biology 26, 179-190.e112, doi:https://doi.org/10.1016/j.chembiol.2018.10.018 (2019).
12 Su, Z. M. et al. Discovery of a Biomarker and Lead Small Molecules to Target r(GGGGCC)-Associated Defects in c9FTD/ALS. Neuron 83, 1043-1050, doi:10.1016/j.neuron.2014.07.041 (2014).
13 Beck, J. et al. Large C9orf72 Hexanucleotide Repeat Expansions Are Seen in Multiple Neurodegenerative Syndromes and Are More Frequent Than Expected in the UK Population. The American Journal of Human Genetics 92, 345-353, doi:https://doi.org/10.1016/j.ajhg.2013.01.011(2013).
14 van Blitterswijk, M. et al. Association between repeat sizes and clinical and pathological characteristics in carriers of C9ORF72 repeat expansions (Xpansize-72): a cross-sectional cohort study. The Lancet Neurology 12, 978-988, doi:https://doi.org/10.1016/S1474-4422(13)70210-2 (2013).
Ash, P. E. et al. Unconventional translation of C9ORF72 GGGGCC expansion generates insoluble polypeptides specific to c9FTD/ALS. Neuron 77, 639-646, doi:10.1016/j.neuron.2013.02.004 (2013).
16 Mori, K. et al. Bidirectional transcripts of the expanded C9orf72 hexanucleotide repeat are translated into aggregating dipeptide repeat proteins. Acta neuropathologica 126, 881-893, doi:10.1007/s00401-013-1189-3 (2013).
17 Mori, K. et al. The C9orf72 GGGGCC repeat is translated into aggregating dipeptide-repeat proteins in FTLD/ALS. Science 339, 1335-1338, doi:10.1126/science.1232927 (2013).
18 Zu, T. et al. RAN proteins and RNA foci from antisense transcripts in C9ORF72 ALS and frontotemporal dementia. Proc Natl Acad Sci USA 110, E4968-4977, doi:10.1073/pnas.1315438110 (2013).
19 Gendron, T. F. et al. Antisense transcripts of the expanded C9ORF72 hexanucleotide repeat form nuclear RNA foci and undergo repeat-associated non-ATG translation in c9FTD/ALS. Acta neuropathologica 126, 829-844, doi:10.1007/s00401-013-1192-8 (2013).
20 Chew, J. et al. Aberrant deposition of stress granule-resident proteins linked to C9orf72-associated TDP-43 proteinopathy. Mol Neurodegener 14, 9, doi:10.1186/s13024-019-0310-z (2019).
21 Gendron, T. F., Belzil, V. V., Zhang, Y. J. & Petrucelli, L. Mechanisms of toxicity in C9FTLD/ALS. Acta neuropathologica 127, 359-376, doi:10.1007/s00401-013-1237-z (2014).
22 Gendron, T. F. et al. Poly(GP) proteins are a useful pharmacodynamic marker for C9ORF72-associated amyotrophic lateral sclerosis. Science translational medicine 9, doi:10.1126/scitranslmed.aai7866 (2017).
23 Han, Y. et al. Structure of Human RNase L Reveals the Basis for Regulated RNA Decay in the IFN Response. Science 343, 1244, doi:10.1126/science.1249845 (2014).
24 Thakur, C. S. et al. Small-molecule activators of RNase L with broad-spectrum antiviral activity. Proceedings of the National Academy of Sciences 104, 9585, doi:10.1073/pnas.0700590104 (2007).
25 Silverman, R. H. Viral encounters with 2′,5′-oligoadenylate synthetase and RNase L during the interferon antiviral response. J Virol 81, 12720-12729, doi:10.1128/jvi.01471-07 (2007).
26 Costales, M. G. et al. Small-molecule targeted recruitment of a nuclease to cleave an oncogenic RNA in a mouse model of metastatic cancer. Proc Natl Acad Sci USA 117, 2406-2411, doi:10.1073/pnas.1914286117 (2020).
27 Costales, M. G., Matsumoto, Y., Velagapudi, S. P. & Disney, M. D. Small Molecule Targeted Recruitment of a Nuclease to RNA. Journal of the American Chemical Society 140, 6741-6744, doi:10.1021/jacs.8b01233 (2018).
28 Costales, M. G., Suresh, B., Vishnu, K. & Disney, M. D. Targeted degradation of a hypoxia-associated non-coding RNA enhances the selectivity of a small molecule interacting with RNA. Cell chemical biology 26, 1180-1186.e1185, doi:10.1016/j.chembiol.2019.04.008 (2019).
29 Liu, X. et al. Targeted Degradation of the Oncogenic MicroRNA 17-92 Cluster by Structure-Targeting Ligands. Journal of the American Chemical Society, doi:10.1021/jacs.9b13159 (2020).
Li, Y. & Disney, M. D. Precise Small Molecule Degradation of a Noncoding RNA Identifies Cellular Binding Sites and Modulates an Oncogenic Phenotype. ACS Chem Biol 13, 3065-3071, doi:10.1021/acschembio.8b00827 (2018).
31 Wang, Z. F. et al. The hairpin form of r(G4C2)exp in c9ALS/FTD is repeat-associated non-ATG translated and a target for bioactive small molecules. Cell chemical biology 26, 179-190.e112, doi:10.1016/j.chembiol.2018.10.018 (2019).
32 Angelbello, A. J. & Disney, M. D. Bleomycin can cleave an oncogenic noncoding RNA. Chembiochem 19, 43-47, doi:10.1002/cbic.201700581 (2018).
33 Moore, S. et al. ADAR2 mislocalization and widespread RNA editing aberrations in C9orf72-mediated ALS/FTD. Acta Neuropathol 138, 49-65, doi:10.1007/s00401-019-01999-w (2019).
34 Zhang, K. et al. The C9orf72 repeat expansion disrupts nucleocytoplasmic transport. Nature 525, 56-61, doi:10.1038/nature14973 (2015).
35 Dobin, A. et al. STAR: ultrafast universal RNA-seq aligner. Bioinformatics 29, 15-21, doi:10.1093/bioinformatics/bts635 (2013).
36 Li, H. et al. The Sequence Alignment/Map format and SAMtools. Bioinformatics 25, 2078-2079, doi:10.1093/bioinformatics/btp352 (2009).
37 Liao, Y., Smyth, G. K. & Shi, W. featureCounts: an efficient general purpose program for assigning sequence reads to genomic features. Bioinformatics 30, 923-930, doi:10.1093/bioinformatics/btt656 (2014).
38 Love, M. I., Huber, W. & Anders, S. Moderated estimation of fold change and dispersion for RNA-seq data with DESeq2. Genome Biol 15, 550, doi:10.1186/s13059-014-0550-8 (2014).
39 Liu, Y. et al. C9orf72 BAC mouse model with motor deficits and neurodegenerative features of ALS/FTD. Neuron 90, 521-534, doi:10.1016/j.neuron.2016.04.005 (2016).
40 Childs-Disney, J. L. et al. Structure of the Myotonic Dystrophy Type 2 RNA and Designed Small Molecules That Reduce Toxicity. ACS Chem. Biol. 9, 538-550, doi:10.1021/cb4007387 (2014).
41 Childs-Disney, J. L. et al. Induction and reversal of myotonic dystrophy type 1 pre-mRNA splicing defects by small molecules. Nat. Commun. 4, doi:10.1038/ncomms3044 (2013).
42 Costales, M. G. et al. Small-molecule targeted recruitment of a nuclease to cleave an oncogenic RNA in a mouse model of metastatic cancer. Proceedings of the National Academy of Sciences 117, 2406, doi:10.1073/pnas.1914286117 (2020).
43 Wang, J. M., Wolf, R. M., Caldwell, J. W., Kollman, P. A. & Case, D. A. Development and testing of a general amber force field (vol 25, pg 1157, 2004). J. Comput. Chem. 26, 114-114, doi:10.1002/jcc.20145 (2005).
44 Bayly, C. I., Cieplak, P., Cornell, W. D. & Kollman, P. A. A well-behaved electrostatic potential based method using charge restraints for deriving atomic charges—the Resp Model. J. Phys. Chem. 97, 10269-10280 (1993).
45 Cornell, W. D., Cieplak, P., Bayly, C. I. & Kollman, P. A. Application of RESP charges to calculate conformational energies, hydrogen-bond energies, and free-energies of solvation. J. Am. Chem. Soc. 115, 9620-9631 (1993).
46 Gaussian 09 v. Revision A.02 (Gaussian, Inc., Wallingford, CT, 2016).
47 Sangster, M. J. L. & Atwood, R. M. Interionic potentials for alkali halides. II. Completely crystal independent specification of Born-Mayer potentials. Journal of Physics C: Solid State Physics 11, 1541-1555, doi:10.1088/0022-3719/11/8/015 (1978).
48 T. E. Cheatham, I., J. L. Miller, T. Fox, T. A. Darden, and P. A. Kollman. Molecular Dynamics Simulations on Solvated Biomolecular Systems: The Particle Mesh Ewald Method Leads to Stable Trajectories of DNA, RNA, and Proteins. J Am Chem Soc 117, 4193-4194 (1995).

### MISCELLANEOUS STATEMENTS

While the invention has been described in conjunction with the detailed description thereof, the foregoing description is intended to illustrate and not limit the scope of the invention, which is defined by the scope of the appended claims. Thus, from the foregoing, it will be appreciated that, although specific nonlimiting embodiments of the invention have been described herein for the purpose of illustration, various modifications may be made without deviating from the spirit and scope of the invention. Other aspects, advantages, and modifications are within the scope of the following claims and the present invention is not limited except as by the appended claims.

The invention has been described broadly and generically herein. Each of the narrower species and subgeneric groupings falling within the generic disclosure also form part of the invention. This includes the generic description of the invention with a proviso or negative limitation removing any patient matter from the genus, regardless of whether or not the excised material is specifically recited herein.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by various nonlimiting embodiments and/or preferred nonlimiting embodiments and optional features, any and all modifications and variations of the concepts herein disclosed that may be resorted to by those skilled in the art are considered to be within the scope of this invention as defined by the appended claims.

All patents, publications, scientific articles, web sites and other documents and material references or mentioned herein are indicative of the levels of skill of those skilled in the art to which the invention pertains, and each such referenced document and material is hereby incorporated by reference to the same extent as if it had been incorporated verbatim and set forth in its entirety herein. The right is reserved to physically incorporate into this specification any and all materials and information from any such patent, publication, scientific article, web site, electronically available information, textbook or other referenced material or document.

All references cited herein are incorporated herein by reference as if fully set forth herein in their entirety.

TABLE 1
Sequences of oligo used in this study.

| | SEQ | |
|---|---|---|
| | ID | |
| Oligo | NO: | Sequence (5′ to 3′) |
| Cy5- | 1 | Cy5-GGGGCCGGGGCCGGGGCCGGG |
| r(G₄C₂)₈ | | GCCGGGGCCGGGGCCGGGGCCGGGG |
| | | CC |
| Cy5- | 2 | Cy5-GGCCGGCCGGCCGGCCGGCCG |
| r(GGCC)₁₀ | | AAAGGCCGGCCGGCCGGCCGGCC |
| FISH Probe | 3 | TYE563 -CCCCGGCCCCGGCCCC- |
| | | TYE563 |

TABLE 2
Sequence of used in this study.

| | SEQ ID | |
|---|---|---|
| Primer | NO: | Sequence (5′ to 3′) |
| GAPDH (fwd) | 4 | GAAGGTGAAGGTCGGAGTC |
| GAPDH (rev) | 5 | GAAGATGGTGATGGGATTTC |
| C9orf72 intron 1 (fwd) | 6 | ACGCCTGCACAATTTCAGCCCAA |
| C9orf72 intron 1 (rev) | 7 | CAAGTCTGTGTCATCTCGGAGCTG |
| C9orf72 exon 2-3 (fwd) | 8 | ACTGGAATGGGGATCGCAGCA |
| C9orf72 exon 2-3 (rev) | 9 | ACCCTGATCTTCCATTCTCTCTGTGCC |
| C9orf72 exon 1b (fwd) | 10 | TGTGACAGTTGGAATGCAGTGA |
| C9orf72 exon 1b (rev) | 11 | GCCACTTAAAGCAATCTCTGTCTTG |
| RNase L (fwd) | 12 | GACACCTCTGCATAACGCAGT |
| RNase L (rev) | 13 | AGGGCTTTGACCTTACCATACA |
| hRRP6 (fwd) | 14 | CTCTTTGGACCTCACGACTGCT |
| hRRP6 (rev) | 15 | AAGAAGCTCGCCTGCTTCTGAA |
| XRN1 (fwd) | 16 | CCAGCAAAGCAGTCGTGGAGAA |
| XRN1 (rev) | 17 | CCACGACTCTAGCTTCCTCAAG |
| XRN2 (fwd) | 18 | CCCAAACCATGTGGTCTTTGTAATC |
| XRN2 (rev) | 19 | TGGTAGGCTGGCCATTGTGA |
| UNKL isoform 1 (fwd) | 20 | CTGCTCCAAGTACAACGAAGCC |
| UNKL isoform 1 (rev) | 21 | TCTGTCTCGTGGATGCAGGTTC |
| Enoyl-CoA (fwd) | 22 | GCTGCCAGCAAGGATGACTCAA |
| Enoyl-CoA (rev) | 23 | GCTTTCTCCTCTACTCCACCAG |
| XYLT1 (fwd) | 24 | TGATGCCTGAGAAGGTGACTCG |
| XYLT1 (rev) | 25 | CACCAGGACAAAGGCGATTCTG |
| RNA BP 10 (fwd) | 26 | GGCATCTACCAACAATCAGCCG |
| RNA BP 10 (rev) | 27 | GGAGAGCAGAACTAGGATGGGT |
| Rab-40C (fwd) | 28 | GTACGCCTACAGTAACGGGATC |
| Rab-40C (rev) | 29 | CTGGAGTAGGACCTGAAGATGG |
| SOCS1 (fwd) | 30 | TTCGCCCTTAGCGTGAAGATGG |
| SOCS1 (rev) | 31 | TAGTGCTCCAGCAGCTCGAAGA |
| USP7 (fwd) | 32 | GTCACGATGACGACCTGTCTGT |
| USP7 (rev) | 33 | GTAATCGCTCCACCAACTGCTG |
| ZFP423 (fwd) | 34 | CTTCTCGCTGGCCTGGGATT |
| ZFP423 (rev) | 35 | GGTCTGCCAGAGACTCGAAGT |
| Mouse β-actin (fwd) | 36 | AGGTATCCTGACCCTGAAG |
| Mouse β-actin (rev) | 37 | GCTCATTGTAGAAGGTGTGG |
| Human C9orf72 insert (fwd) | 38 | TCTCCAGCTGTTGCCAAGAC |
| Human C9orf72 insert (rev) | 39 | TCCATTCTCTCTGTGCCTTCT |

TABLE 3
Sequences of ASOs and siRNA used
in this study.

| | SEQ | |
|---|---|---|
| | ID | |
| Name | NO | Sequence (5′ to 3′)^{a} |
| G4C2-ASO | 40 | mG*mG*mC*C*C*C*G*G*C*C*C*C*G*G* |
| | | C*C*C*mC*mG*mG |
| C9orf72-ASO | 41 | mU*mA*mC*A*G*G*C*T*G*C*G*G*T*T* |
| | | G*T*T*mU*mC*mC |
| Control-ASO | 42 | mC*mC*mU*T*C*C*C*T*G*A*A*G*G*T* |
| RNase L | | T*C*C*mU*mC*mC |
| siRNA | | |
| hRRP6 siRNA | 43 | GCAAAAUCUGAAACUUUCCdTdT |
| XRN1 siRNA | | |
| XRN2 siRNA | | |

^{a}m indicates 2′-O-methyl residue;
*indicates LNA residue; miRUCRY LNA (Qiagen);
siRNAs were purchased from Horizon Discovery Biosciences

TABLE 4
Sex, genotype, and age of mice used in in vivo studies.

| | | | | |
|---|---|---|---|---|
| Mouse | Sex | Genotype | Age | Treatment |

3 Week Treatment Period

| | | | | |
|---|---|---|---|---|
| 1 | F | (+) | 21 | Vehicle |
| 2 | M | (+) | 20 | Vehicle |
| 3 | M | (+) | 19 | Vehicle |
| 4 | F | (+) | 19 | Vehicle |
| 5 | F | (+) | 18 | Vehicle |
| 6 | F | (+) | 18 | Vehicle |
| 7 | F | (+) | 20 | 33 nmol 2 |
| 8 | M | (+) | 20 | 33 nmol 2 |
| 9 | F | (+) | 20 | 33 nmol 2 |
| 10 | M | (+) | 18 | 33 nmol 2 |
| 11 | F | (+) | 18 | 33 nmol 2 |
| 12 | M | (+) | 18 | 33 nmol 2 |
| 13 | F | (−) | 20 | Vehicle |
| 14 | M | (−) | 19 | Vehicle |
| 15 | F | (−) | 19 | Vehicle |
| 16 | M | (−) | 18 | Vehicle |
| 17 | F | (−) | 20 | 33 nmol 2 |
| 18 | M | (−) | 19 | 33 nmol 2 |
| 19 | F | (−) | 19 | 33 nmol 2 |
| 20 | M | (−) | 18 | 33 nmol 2 |

TABLE 5
Summary of cell line demographic information and the
corresponding figures in which they were used.

| | Cell | | | | | |
|---|---|---|---|---|---|---|
| Identifier | Type | Diagnosis | Sex | Age | Source | Experiments depicted by FIG. |
| CRL- | HEK293T | Control | Female | Fetus | ATCC | 7 |
| 3216 | | | | | | |
| GM07491 | LCL | Healthy | Male | 17 | Coriell | 11A |
| ND11583 | LCL | C9orf72 | Male | 59 | Coriell | 2B, 2D, 2E, 10A |
| ND09492 | LCL | C9orf72 | Male | 52 | Coriell | 2B, 2D, |
| ND12438 | LCL | C9orf72 | Male | 65 | Coriell | 2B, 2D, |
| CS9XH7 | iPSC | Healthy | Male | 53 | Cedars | 11C-11E |
| | | | | | Sinai | |
| CS8PAA | iPSC | Healthy | Female | 58 | Cedars | 11C-11E, 14B-11D |
| | | | | | Sinai | |
| CS0002 | iPSC | Healthy | Male | 51 | Cedars | 11C-11E, 12D-F |
| | | | | | Sinai | |
| EDi044-A | iPSC | Healthy | Female | 80 | Cedars | 11C-11E |
| | | | | | Sinai | |
| CS7VCZ | iPSC | C9orf72 | Male | 64 | Cedars | 3A-F, 4B-C, 10B-C, 11B, 11F-G, |
| | | | | | Sinai | 12A-C, 13, 14A, 15 |
| CS0BUU | iPSC | C9orf72 | Female | 63 | Cedars | 3A-3C, 11B, 11F |
| | | | | | Sinai | |
| CS2YNL | iPSC | C9orf72 | Male | 60 | Cedars | 3A-3C, 11B |
| | | | | | Sinai | |
| CS0NKC | iPSC | C9orf72 | Female | 60 | Cedars | 3A-3C, 11B |
| | | | | | Sinai | |

What is claimed is:

1. A method comprising contacting a hexanucleotide repeat expansion RNA r(G₄C₂)^{exp} with an ALS compound wherein the ALS compound comprises a pyridocarbazole moiety bound to an RNase moiety according to Formula I 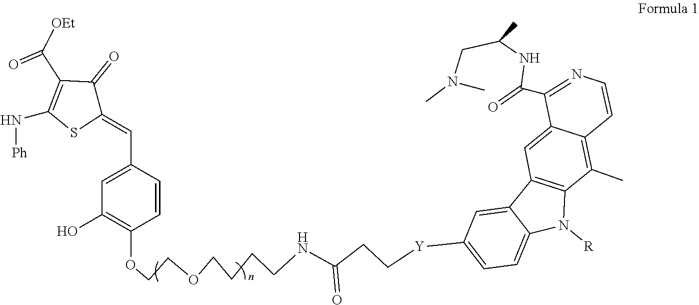
wherein:
R is hydrogen or a C1-C3 alkyl group, preferably a methyl group, more preferably hydrogen,
Y is —COO, —CH₂—O—, preferably —CH₂—O—
n is an integer of 1 to 6, preferably 2-4, more preferably 2,
and a pharmaceutically acceptable salt thereof.

2. A method according to claim 1 wherein the contacting binds and/or complexes the r(G₄C₂)^{exp}.

3. A method according to any of claims 1-2 wherein the r(G₄C₂)^{exp} is r(G₄C₂)ₘ wherein m is at least 20.

4. A method according to any of the preceding claims wherein r(G₄C₂)ₘ is an abnormal number of repeats with m being at least 20-1000.

5. A method according to any of the preceding claims wherein the r(G₄C₂)^{exp} is a repeat RNA hairpin structure.

6. A method according to any of preceding claims wherein r(G₄C₂)^{exp} is present in a cell.

7. A method according to claim 6 wherein the cell contains chromosome 9 open reading frame 72 (C9orf72) and r(G₄C₂)^{exp} is present in the intron 1 of C9orf72.

8. A method according to any of claims 6 and 7 wherein the cells are patient-derived cells.

9. A method of any of claims 6-8 wherein the cells are incubated with the ALS compound of claim 1.

10. A method according to claim 9 wherein the cells are HEK293T cells, patient-derived lymphoblastoid cells, induced pluripotent stem cells (c9 iPSCs cells), iPSC-derived spinal neurons (c9 iPSNs).

11. A method according to claim 10 wherein the cells are c9ALS/FTD BAC cells in a transgenic mouse model.

12. A method according to any of the preceding claims 6-11 wherein the ALS compound decreases RAN translation of r(G₄C₂)^{exp}.

13. A method according to claim 12 wherein the ALS compound inhibits RAN translation of r(G₄C₂)^{exp}.

14. A method according to any of the preceding claims 6-13 wherein the ALS compound does not inhibit transcription of C9orf72.

15. A method according to any of the preceding claims 6-14 wherein the ALS compound decreases the number of nuclear foci.

16. A method according to any the preceding claims 6-15 wherein the ALS compound alleviates defects in nuclear trafficking.

17. A method according to any of the preceding claims 6-16 wherein the ALS compound facilitates degradation of the repeat expansion.

18. A pharmaceutical composition comprising a pharmaceutically acceptable carrier and an ALS compound according to Formula I of claim 1.

19. A pharmaceutical composition according to any of claim 18 wherein the pharmaceutically acceptable carrier comprises excipients suitable for a selected route of administration of the ALS compound.

20. A pharmaceutical composition according to any of claims 18-19 wherein the amount of ALS compound provides an effective dose of the ALS compound for treatment of a disease caused by G₄C₂ repeat expansion, preferably ALS/FTD disease.

21. A method for treatment of an ALS/FTD disease comprising administration to a patient having the disease, an effective amount of an ALS compound of claim 1.

22. A method for treatment of an ALS/FTD disease comprising administration to a patient having the disease, a pharmaceutical composition of any of claims 18-20.

23. A method for treatment according to any of claims 21-22 wherein the ALS/FTD disease is amyotrophic lateral sclerosis.

24. A method according to claim 23 wherein the administration step comprises oral, intramuscular, intravenous or intrathecal administration of the ALS compound in a pharmaceutically acceptable medium.

25. A method according to claim 24 wherein the ALS compound in a pharmaceutically acceptable medium is a pharmaceutical composition.

26. A method according to claim 25 wherein the pharmaceutical composition comprises pharmaceutically acceptable excipients suitable for a selected route of administration and the excipients are compatible with the ALS compound.

27. A composition comprising an ALS compound of Formula I and a pharmaceutically acceptable salt thereof: 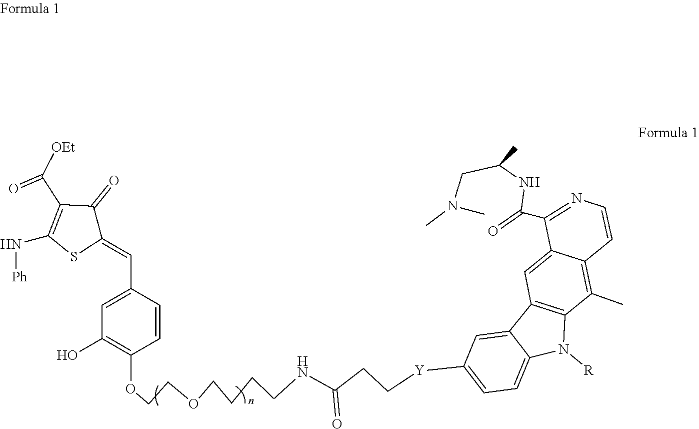
wherein:
R is hydrogen or a C1-C3 alkyl group, preferably a methyl group, more preferably hydrogen,
Y is —COO— or —CH₂—O—, preferably —CH₂—O—
n is an integer of 1 to 6, preferably 2-4, more preferably 2,
and a pharmaceutically acceptable salt thereof.


SEQUENCE LISTING (49 sequences)

SEQ ID NO: 1 - DNA, 48 bp, synthetic construct
  source 1..48: mol_type = other DNA
ggggccggggccggggccggggccggggccggggccggggccggggcc

SEQ ID NO: 2 - DNA, 44 bp, synthetic construct
  source 1..44: mol_type = other DNA
ggccggccggccggccggccgaaaggccggccggccggccggcc

SEQ ID NO: 3 - DNA, 16 bp, synthetic construct
  source 1..16: mol_type = other DNA
ccccggccccggcccc

SEQ ID NO: 4 - DNA, 19 bp, synthetic construct
  source 1..19: mol_type = other DNA
gaaggtgaaggtcggagtc

SEQ ID NO: 5 - DNA, 20 bp, synthetic construct
  source 1..20: mol_type = other DNA
gaagatggtgatgggatttc

SEQ ID NO: 6 - DNA, 23 bp, synthetic construct
  source 1..23: mol_type = other DNA
acgcctgcacaatttcagcccaa

SEQ ID NO: 7 - DNA, 24 bp, synthetic construct
  source 1..24: mol_type = other DNA
caagtctgtgtcatctcggagctg

SEQ ID NO: 8 - DNA, 21 bp, synthetic construct
  source 1..21: mol_type = other DNA
actggaatggggatcgcagca

SEQ ID NO: 9 - DNA, 27 bp, synthetic construct
  source 1..27: mol_type = other DNA
accctgatcttccattctctctgtgcc

SEQ ID NO: 10 - DNA, 22 bp, synthetic construct
  source 1..22: mol_type = other DNA
tgtgacagttggaatgcagtga

SEQ ID NO: 11 - DNA, 25 bp, synthetic construct
  source 1..25: mol_type = other DNA
gccacttaaagcaatctctgtcttg

SEQ ID NO: 12 - DNA, 21 bp, synthetic construct
  source 1..21: mol_type = other DNA
gacacctctgcataacgcagt

SEQ ID NO: 13 - DNA, 22 bp, synthetic construct
  source 1..22: mol_type = other DNA
agggctttgaccttaccataca

SEQ ID NO: 14 - DNA, 22 bp, synthetic construct
  source 1..22: mol_type = other DNA
ctctttggacctcacgactgct

SEQ ID NO: 15 - DNA, 22 bp, synthetic construct
  source 1..22: mol_type = other DNA
aagaagctcgcctgcttctgaa

SEQ ID NO: 16 - DNA, 22 bp, synthetic construct
  source 1..22: mol_type = other DNA
ccagcaaagcagtcgtggagaa

SEQ ID NO: 17 - DNA, 22 bp, synthetic construct
  source 1..22: mol_type = other DNA
ccacgactctagcttcctcaag

SEQ ID NO: 18 - DNA, 25 bp, synthetic construct
  source 1..25: mol_type = other DNA
cccaaaccatgtggtctttgtaatc

SEQ ID NO: 19 - DNA, 20 bp, synthetic construct
  source 1..20: mol_type = other DNA
tggtaggctggccattgtga

SEQ ID NO: 20 - DNA, 22 bp, synthetic construct
  source 1..22: mol_type = other DNA
ctgctccaagtacaacgaagcc

SEQ ID NO: 21 - DNA, 22 bp, synthetic construct
  source 1..22: mol_type = other DNA
tctgtctcgtggatgcaggttc

SEQ ID NO: 22 - DNA, 22 bp, synthetic construct
  source 1..22: mol_type = other DNA
gctgccagcaaggatgactcaa

SEQ ID NO: 23 - DNA, 22 bp, synthetic construct
  source 1..22: mol_type = other DNA
gctttctcctctactccaccag

SEQ ID NO: 24 - DNA, 22 bp, synthetic construct
  source 1..22: mol_type = other DNA
tgatgcctgagaaggtgactcg

SEQ ID NO: 25 - DNA, 22 bp, synthetic construct
  source 1..22: mol_type = other DNA
caccaggacaaaggcgattctg

SEQ ID NO: 26 - DNA, 22 bp, synthetic construct
  source 1..22: mol_type = other DNA
ggcatctaccaacaatcagccg

SEQ ID NO: 27 - DNA, 22 bp, synthetic construct
  source 1..22: mol_type = other DNA
ggagagcagaactaggatgggt

SEQ ID NO: 28 - DNA, 22 bp, synthetic construct
  source 1..22: mol_type = other DNA
gtacgcctacagtaacgggatc

SEQ ID NO: 29 - DNA, 22 bp, synthetic construct
  source 1..22: mol_type = other DNA
ctggagtaggacctgaagatgg

SEQ ID NO: 30 - DNA, 22 bp, synthetic construct
  source 1..22: mol_type = other DNA
ttcgcccttagcgtgaagatgg

SEQ ID NO: 31 - DNA, 22 bp, synthetic construct
  source 1..22: mol_type = other DNA
tagtgctccagcagctcgaaga

SEQ ID NO: 32 - DNA, 22 bp, synthetic construct
  source 1..22: mol_type = other DNA
gtcacgatgacgacctgtctgt

SEQ ID NO: 33 - DNA, 22 bp, synthetic construct
  source 1..22: mol_type = other DNA
gtaatcgctccaccaactgctg

SEQ ID NO: 34 - DNA, 20 bp, synthetic construct
  source 1..20: mol_type = other DNA
cttctcgctggcctgggatt

SEQ ID NO: 35 - DNA, 21 bp, synthetic construct
  source 1..21: mol_type = other DNA
ggtctgccagagactcgaagt

SEQ ID NO: 36 - DNA, 19 bp, synthetic construct
  source 1..19: mol_type = other DNA
aggtatcctgaccctgaag

SEQ ID NO: 37 - DNA, 20 bp, synthetic construct
  source 1..20: mol_type = other DNA
gctcattgtagaaggtgtgg

SEQ ID NO: 38 - DNA, 20 bp, synthetic construct
  source 1..20: mol_type = other DNA
tctccagctgttgccaagac

SEQ ID NO: 39 - DNA, 21 bp, synthetic construct
  source 1..21: mol_type = other DNA
tccattctctctgtgccttct

SEQ ID NO: 40 - DNA, 20 bp, synthetic construct
  source 1..20: mol_type = other DNA
  misc_feature 1..3: note = RNA
  misc_feature 4..17: note = DNA
  misc_feature 18..20: note = RNA
  modified_base 1..3: mod_base = OTHER; note = 2-O-methyl nucleosides
  modified_base 18..20: mod_base = OTHER; note = 2-O-methyl nucleosides
  modified_base 2..20: mod_base = OTHER; note = locked nucleic acids (contain 2'-O, 4'-C methylene bridge)
ggccccggccccggccccgg

SEQ ID NO: 41 - DNA, 20 bp, synthetic construct
  source 1..20: mol_type = other DNA
  misc_feature 1..3: note = RNA
  misc_feature 18..20: note = RNA
  misc_feature 4..17: note = DNA
  modified_base 1..3: mod_base = OTHER; note = 2-O-methyl nucleosides
  modified_base 18..20: mod_base = OTHER; note = 2-O-methyl nucleosides
  modified_base 2..20: mod_base = OTHER; note = locked nucleic acids (contain 2'-O, 4'-C methylene bridge)
  modified_base 1: mod_base = OTHER; note = uracil
  modified_base 17: mod_base = OTHER; note = uracil
tacaggctgcggttgtttcc

SEQ ID NO: 42 - DNA, 20 bp, synthetic construct
  source 1..20: mol_type = other DNA
  misc_feature 1..3: note = RNA
  misc_feature 18..20: note = RNA
  misc_feature 4..17: note = DNA
  modified_base 1..3: mod_base = OTHER; note = 2-O-methyl nucleosides
  modified_base 18..20: mod_base = OTHER; note = 2-O-methyl nucleosides
  modified_base 2..20: mod_base = OTHER; note = locked nucleic acids (contain 2'-O, 4'-C methylene bridge)
  modified_base 3: mod_base = OTHER; note = uracil
  modified_base 17: mod_base = OTHER; note = uracil
ccttccctgaaggttcctcc

SEQ ID NO: 43 - RNA, 21 nt, synthetic construct
  source 1..21: mol_type = other RNA
  modified_base 20..21: mod_base = OTHER; note = deoxythymidine
gcaaaatctgaaactttcctt

SEQ ID NO: 44 - DNA, 48 bp, synthetic construct; identical to SEQ ID NO: 1
  source 1..48: mol_type = other DNA

SEQ ID NO: 45 - DNA, 396 bp, synthetic construct
  source 1..396: mol_type = other DNA
ggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggcc

SEQ ID NO: 46 - DNA, 48 bp, synthetic construct; identical to SEQ ID NO: 1
  source 1..48: mol_type = other DNA

SEQ ID NO: 47 - DNA, 32 bp, synthetic construct
  source 1..32: mol_type = other DNA
ggccggccggccggccggccggccggccggcc

SEQ ID NO: 48 - DNA, 24 bp, synthetic construct
  source 1..24: mol_type = other DNA
ggggccggggccggggccggggcc

SEQ ID NO: 49 - DNA, 44 bp, synthetic construct
  source 1..44: mol_type = other DNA
ggggccggggccggggccggggccgaaaggccggccggccggcc